(19) United States
(12) Patent Application Publication
(10) Pub. No.: US 2022/0143166 A1
(43) Pub. Date: May 12, 2022

(54) MULTIVALENT PNEUMOCOCCAL POLYSACCHARIDE-PROTEIN CONJUGATE VACCINE

(71) Applicant: Biological E Limited, Hyderabad, Telangana (IN)

(72) Inventors: Rajendar BURKI, Hyderabad, Telangana (IN); Rajan SRIRAMAN, Hyderabad, Telangana (IN); Ramesh Venkat MATUR, Hyderabad, Telangana (IN); Narender Dev MANTENA, Hyderabad, Telangana (IN); Mahima DATLA, Hyderabad, Telangana (IN); Balamurali MASILAMANI, Hyderabad, Telangana (IN); Vivek Babu KANDIMALLA, Hyderabad, Telangana (IN); Veerapandu SANGAREDDY, Hyderabad, Telangana (IN)

(21) Appl. No.: 17/312,820

(22) PCT Filed: Oct. 11, 2019
(86) PCT No.: PCT/IN2019/050761

§ 371 Date: Jun. 10, 2021

(30) Foreign Application Priority Data
Oct. 12, 2018 (IN) 201841038835

(51) Int. Cl. A61K 39/09

(52) U.S. Cl. CPC A61K 39/092; A61K 2039/70

(57) ABSTRACT

The present invention relates to multivalent pneumococcal polysaccharide-protein conjugates vaccine composition comprising pneumococcal capsular polysaccharide of one or more Streptococcus pneumoniae serotypes conjugated to one or more carrier proteins.

### FIELD OF THE INVENTION

The present invention relates to multivalent pneumococcal polysaccharide-protein conjugates vaccine composition comprising pneumococcal capsular polysaccharide of one or more Streptococcus pneumoniae serotypes conjugated to one or more carrier proteins.

### BACKGROUND OF THE INVENTION

Streptococcus pneumoniae (“pneumococcus”) is a gram-positive bacterium that causes invasive diseases, such as pneumonia, bacteremia and meningitis, and diseases associated with colonization, such as acute otitis media (e.g., colonization of middle ear). These pneumococcus-induced diseases result in morbidity and mortality, particularly in persons less than 24 months old and greater than 60 years old. The rate of pneumococcal pneumonia in the U.S. for persons over 60 years of age is estimated to be 3 to 8 per 100,000. In 20% of cases, pneumococcal pneumonia leads to bacteremia and meningitis collectively having a mortality rate close to 30% despite antibiotic treatment.

Pneumococcal vaccines may be administered to prevent infections. Current vaccines include multivalent pneumococcal polysaccharide vaccines (comprises pneumococcal polysaccharides from two or more serotypes) and pneumococcal conjugate vaccines. The protective efficacy of the pneumococcal polysaccharide vaccine is known to be related to the concentration of antibody generated against a capsular polysaccharide. Pneumococcus cells are encapsulated with a polysaccharide giving rise to more than 90 different pneumococcus serotypes. The capsule is the principal virulence determinant for pneumococci—it not only protects the cell's inner surface from complement mediated cell lysis, it is also poorly immunogenic.

Merck's Pneumovax®23 is a multivalent pneumococcal polysaccharide vaccine and contains unconjugated capsular polysaccharides from 23 pneumococcal serotypes including serotypes 1, 2, 3, 4, 5, 6B, 7F, 8, 9N, 9V, 10A, 11A, 12F, 14, 15B, 17F, 18C, 19F, 19A, 20, 22F, 23F and 33F. In addition to Pneumavax®23, the multivalent pneumococcal polysaccharide vaccines that have been licensed so far proved valuable in preventing pneumococcal disease in adults, particularly, the elderly and those at high-risk. However, infants and young children respond poorly to these unconjugated pneumococcal polysaccharide vaccines.

Prevnar®-7 is a pneumococcal polysaccharide-protein conjugate vaccine and includes the seven most frequently isolated polysaccharide serotypes (e.g., 4, 6B, 9V, 14, 18C, 19F, and 23F conjugated to CRM₁₉₇). Since the use of Prevnar®-7 began in the United States in 2000, there has been a significant reduction in invasive pneumococcal disease (IPD) in children. A 13-valent conjugate vaccine Prevenar-13®, containing thirteen serotypes 1, 3, 4, 5, 6A, 6B, 7F, 9V, 14, 18C, 19A, 19F and 23F conjugated to CRM₁₉₇, was developed and approved due to the limitations in serotype coverage with Prevnar®-7 in certain regions of the world.

Synflorix® is a pneumococcal vaccine that includes ten polysaccharide serotypes 4, 5, 6B, 7F, 9V, 14, 23F conjugated to protein D (PD), serotype 18C conjugated to tetanus toxoid (TT) and serotype 19F conjugated to diphtheria toxoid (DT). Each of the serotype polysaccharides is coupled utilizing 1-cyano-4-dimethylamino-pyridinium tetrafluoroborate (CDAP) under controlled pH.

U.S. Pat. No. 5,360,897 discloses pneumococcal vaccines wherein an immunogenic conjugate comprising a reductive amination product of an intact capsular polymer of the bacterial pathogen Streptococcus pneumoniae having at least two carbonyl groups and a bacterial toxin or toxoid, said conjugate comprising a cross-linked conjugate in which there is a direct covalent linkage between the capsular polymer and the toxin or toxoid.

U.S. Pat. No. 5,693,326 provides a generalized method for preparing a conjugate vaccine wherein for activating viral, fungal or bacterial polysaccharides, an organic cyanylating agent is used selected from the group 1-cyano-4-(dimethylamino)-pyridinium tetrafluoroborate, N-cyanotriethyl-ammonium tetrafluoroborate, and p-nitrophenylcyanate, to form an activated carbohydrate and is subsequently coupled to the protein or carrier protein.

U.S. Pat. No. 5,854,416 discloses amino acid and DNA sequences of 37-kDa protein from Streptococcus pneumonia known as PsaA (Pneumococcal surface adhesion A).

U.S. Pat. No. 7,862,823 discloses a multivalent conjugate vaccine composition comprising pneumococcal capsular polysaccharides with at least two different carrier proteins, such as DT and TT.

U.S. Pat. No. 8,192,746 discloses a 15-valent pneumococcal polysaccharide-protein conjugate vaccine composition having capsular polysaccharides from serotypes 1, 3, 4, 5, 6A, 6B, 7F, 9V, 14, 18C, 19A, 19F, 22F, 23F, and 33F conjugated to CRM₁₉₇.

U.S. Pat. No. 8,557,250 B2 discloses a method comprising contacting a mixture of a plurality of cyanate activated immunogenic distinct polysaccharides with at least one hydrazide activated protein.

U.S. Pat. Nos. 8,808,708 and 8,603,484 describes a 13-valent immunogenic composition consisting of polysaccharide-protein conjugates wherein serotypes consist of 1, 3, 4, 5, 6A, 611, 7F, 9V, 14, 18C, 19A, 19F and 23F and carrier protein CRM₁₉₇.

U.S. Patent Publication No. 2010/0074922 A1 discloses an immunogenic composition containing 10 or more serotypes wherein 19F capsular saccharide is conjugated to DT, serotype 18C capsular saccharide is conjugated to tetanus toxoid and serotypes 1, 4, 5, 6B, 7F, 9V, 14 and 23F capsular saccharides are conjugated to Protein D isolated from Haemophilus influenzae.

U.S. Patent Publication No. 2010/0239604 describes an immunogenic composition comprising multivalent Streptococcus pneumoniae capsular saccharide conjugates from serotypes 19A and 19F wherein serotype 19A is conjugated to a first bacterial toxoid and 19F is conjugated to a second bacterial toxoid and 2-9 of the Streptococcus pneumoniae capsular saccharides are conjugated to protein D.

U.S. Patent Publication No. 2012/321658 A1 discloses an immunogenic composition wherein serotypes 1, 3, 19A and 19F linked to protein carrier(s) either directly or indirectly through a chemistry other than reductive amination, and one or more different saccharides is/are selected from a second group consisting of serotypes 4, 5, 6A, 6B, 7F, 9V, 14, 18C and 23F which is/are linked to a protein carrier(s) by reductive amination.

IN 140/DEL/2011 provides a Streptococcus pneumonia vaccine comprising either of (a) 7 or more (b) 10 or more polysaccharides from serotypes conjugated to at least 2 or more carrier proteins selected from a group comprising DT, diphtheria toxoid. CRM₁₉₇, and tetanus toxoid.

WO Publication No. 2013/191459 A1 discloses a 15 valent pneumococcal conjugate composition comprising different serotypes of Streptococcus pneumoniae derived from a capsular polysaccharide 1, 2, 3, 4, 5, 6A, 6B, 7F, 9N, 9V, 14, 18C, 19A, 19F and 23F conjugated to CRM₁₉₇.

WO Publication No. 2014/092377 A1 discloses a 13 valent pneumococcal conjugate composition wherein 12 serotypes are selected from 1, 3, 4, 5, 6A, 6B, 7F, 9V, 14, 18C, 19A, 19F. and 23F and the last serotype is either 2 or 9N conjugated to CRM₁₉₇.

WO Publication No. 2014/092378 A1 describes an immunogenic pneumococcal conjugate composition where 12 serotypes are selected from 1, 3, 4, 5, 6A, 6B, 7F, 9V, 14, 18C, 19A, 19F, and 23F and remaining one from 22F or 33F conjugated to CRM₁₉₇.

WO Publication No. 2016/207905 A2 discloses a multivalent pneumococcal conjugate vaccine (PCV) composition comprising: 1) at least 12 capsular polysaccharides selected from serotypes 1, 3, 4, 5, 6B, 7F, 9N, 9V, 15B, 14, 18C, 19A, 19F, 22F, 23F and 33F of Streptococcus pneumoniae activated with CDAP and conjugated to carrier protein CRM₁₉₇, and 2) a pharmaceutically acceptable carrier, wherein the composition does not contain capsular polysaccharide from serotype 6A.

WO 2018/064444A1 of the present applicant describes a pneumococcal vaccine composition, the composition comprising two or more capsular pneumococcal polysaccharide serotypes each individually conjugated to a carrier protein pneumococcal surface adhesion protein A (PsaA) or combination of PsaA and CRM₁₉₇.

Chinese Patent Application Publication No. CN 101590224 describes a 14 valent pneumococcal polysaccharide-protein conjugate vaccine containing serotypes 1, 2, 4, 5, 6A, 6B, 7F, 9N, 9V, 14, 18C, 19A, 19F and 23F conjugated to CRM₁₉₇.

Chinese Patent Application Publication No. CN 103623401 discloses a 14 valent pneumococcal capsular polysaccharide-protein conjugate composition wherein said 14 different serotypes are 1, 3, 4, 5, 6A, 6B, 9V, 14,18C, 19A, 19F, 22F, 23F and 33F conjugated to CRM₁₉₇.

Chinese Patent Application Publication No. CN 103656631 provides a multivalent pneumococcus capsular polysaccharide-protein conjugate composition and a preparation method thereof. The conjugate composition is prepared from capsular polysaccharides of pneumococcus of 24 different serotypes and a carrier protein in a covalent linkage manner, wherein the 24 different serotypes are 1, 2, 3, 4, 5, 6A, 6B, 7F, 8, 9N, 9V, 10A, 11A, 12F, 14, 15B, 17F, 18C, 19A, 19F, 20, 22F, 23F and 33F conjugated to CRM₁₉₇.

Chinese Patent Application Publication No, CN 103656632 discloses a multivalent pneumococcal capsular polysaccharide composition containing serotype 6A and at least one extra serotype selected from the group consisting of 1, 2, 3, 4; 5, 6B, 7F, 8, 9N, 10A, 11A, 12F, 14, 15B, 17F, 18C, 19A, 19F. 20, 22F, 23F and 33F conjugated to CRM₁₉₇.

Chinese Patent Application Publication No. CN 104069488 discloses a multivalent pneumococcus capsular polysaccharide vaccine of 14 different serotypes and carrier protein, wherein the 14 serotypes include 1, 4, 5, 6A, 6B, 7F, 9V, 14, 18C, 19A, 19F, 22F, 23F and 33F conjugated to CRM₁₉₇.

Anderson P et al, (2003, Vaccine; 21 (13-14):1554-9) discloses a comparative study of tetravalent pneumococcal conjugate vaccines with each polysaccharide types 6A, 14, 19F, and 23F separately coupled to tetanus toxoid or diphtheria CRM₁₉₇ or a mixture of halved doses of polysaccharide types 6A, 14, 19F, and 23F separately coupled to tetanus toxoid and diphtheria CRM₁₉₇.

Nurkka et al. (2004, Ped. Inf Dis. 1, 23:1008-1014) discloses a study of the immunogenicity and safety of an 11-valent pneumococcal protein D conjugate vaccine where no priming effect was observed for serotype 3 in infants who had received three doses of the vaccine followed by a booster dose of either the same vaccine or a pneumococcal polysaccharide vaccine.

The above-mentioned references disclose, amongst other compositions, methods, and the like, multivalent pneumococcal conjugate vaccines comprising polysaccharides from one or more serotypes as well as conjugation of these polysaccharides with carrier proteins. In view of the different serotypes that are prevalent across various regions, there is a need for additional multivalent pneumococcal vaccines comprising novel conjugates of polysaccharide serotypes with improved immune response, as well as to develop simple and efficient production thereof.

Surprisingly, the multivalent pneumococcal conjugate vaccine compositions of the present invention offer an improved immune response over the naive multivalent pneumococcal vaccines and existing pneumococcal conjugate vaccines.

### SUMMARY OF THE INVENTION

The present invention provides a 24-valent pneumococcal polysaccharide protein conjugate vaccine composition comprising one or more Streptococcus pneumoniae serotypes conjugated to one or more carrier protein(s).

In another embodiment, the present invention provides a pneumococcal conjugate vaccine composition comprising capsular polysaccharide from serotypes of Streptococcus pneumoniae conjugated to one or more carrier proteins, wherein the serotypes comprise 1, 3, 4, 5, 6A, 6B, 7F, 8, 9V, 10A, 1.1 A, 12F, 14, 15A, 18C, 19A, 19F, 22F, 23A, 23B, 23F, 24F, 33F and 35B.

In yet another embodiment, the present invention also provides a pneumococcal conjugate vaccine composition comprising capsular polysaccharide from serotypes of Streptococcus pneumoniae conjugated to one or more carrier proteins, wherein the serotypes comprises of 1, 3, 4, 5, CA, 6B, 7F, 8, 9V, 10A, 11A, 12F, 14, 15A, 18C, 19A, 19F, 22F, 23A, 23B, 23F, 24F, 33F and 35B and the carrier protein is selected from CRM₁₉₇ or combination of CRM₁₉₇ and PsaA or combination of CRM₁₉₇ and Tetanus toxoid or combination of PsaA and Tetanus toxoid or combination of CRM₁₉₇, PsaA and Tetanus toxoid or combination of CRM₁₉₇, PsaA, Protein D, Diphtheria toxic and Tetanus toxoid.

In an embodiment, the present invention provides a pneumococcal conjugate vaccine composition comprising capsular polysaccharide from serotypes of Streptococcus pneumoniae conjugated to a carrier protein, wherein the serotypes comprise 1, 3, 4, 5, 6A, 6B, 7F, 8, 9V, 10A, 11A, 12F, 14, 15A, 18C, 19A, 19F, 22F, 23A, 23B, 23F, 24F, 33F and 35B and the carrier protein is CRM₁₉₇.

In an embodiment, the present invention provides a pneumococcal conjugate vaccine composition comprising capsular polysaccharide from serotypes of Streptococcus pneumoniae conjugated to a carder protein, wherein the serotypes comprise 1, 3, 4, 5, 6A, 6B, 7F, 8, 9V, 10A, 11A, 12F, 14, 15A, 18C, 19A, 19F, 22F, 23A, 23B, 23F, 24F, 33F and 35B and the carrier protein is PsaA.

In an embodiment, the present invention provides a pneumococcal conjugate vaccine composition comprising capsular polysaccharide from serotypes of Streptococcus pneumoniae conjugated to one or more carrier proteins, wherein the serotypes comprise 1, 3, 4, 5, 6A, 6B, 7F, 8, 9V, 10A, 11A, 12F, 14, 15A, 18C, 19A, 19F, 22F, 23A, 23B, 23F, 24F, 33F and 35B and the carrier protein is CRM₁₉₇, PsaA or combination thereof.

In an embodiment, the present invention provides a pneumococcal conjugate vaccine composition comprising capsular polysaccharide from serotypes of Streptococcus pneumoniae conjugated to one or more carrier proteins, wherein the serotypes comprise 1, 3, 4, 5, 6A, 6B. 7F. 8, 9V, 10A, 11A, 12F, 14, 15A, 18C, 19A, 19F, 22F, 23A, 23B, 23F, 24F, 33F and 35B and the carrier protein is CRM₁₉₇, PsaA, Tetanus toxoid or combination thereof.

### BRIEF DESCRIPTION OF THE DRAWINGS

FIG. 1 SEC-HPLC chromatogram illustrating conjugation reaction kinetics of (A) serotype 3 and (B) serotype 6B with PsaA,

FIG. 2 SEC-HPLC chromatogram illustrating conjugation reaction kinetics of (A) serotype 6A-CRM₁₉7 and (B) serotype 6A with PsaA.

FIG. 3 SEC-HPLC chromatogram illustrating conjugation reaction kinetics of (A) serotype 8-CRM₁₉₇ and (B) serotype 8-PsaA.

FIG. 4 SEC-HPLC chromatogram illustrating conjugation reaction kinetics of (A) serotype 10A-CRM₁₉₇ and (B) serotype 10A-PsaA.

FIG. 5 SEC-HPLC chromatogram illustrating conjugation reaction kinetics of (A) serotype 11A-CRM₁₉₇ and (B) serotype 11A-PsaA.

FIG. 6 SEC-HPLC chromatogram illustrating conjugation reaction kinetics of (A) serotype 12F-CRM₁₉₇ and (B) serotype 12F-PsaA.

FIG. 7 SEC-HPLC chromatogram illustrating conjugation reaction kinetics of (A) serotype 15A-CRM₁₉₇ and (B) serotype 15A-PsaA

FIG. 8 SEC-HPLC chromatogram illustrating conjugation reaction kinetics of (A) serotype 23A-CRM₁₉₇ and (B) serotype 23A-PsaA.

FIG. 9 SEC-HPLC chromatogram illustrating conjugation reaction kinetics of (A) serotype 23B-CRM₁₉₇ and (b) serotype 23B-PsaA.

FIG. 10 SEC-HPLC chromatogram illustrating conjugation reaction kinetics of (A) serotype 24F-CRM₁₉₇ and (B) serotype 24F-PsaA.

FIG. 11 SEC-HPLC chromatogram illustrating conjugation reaction kinetics of (A) serotype 35B-CRM₁₉₇ and (B) serotype 35B-PsaA.

FIG. 12A: Serum antibody titers in rabbits immunized with Formulation I

FIG. 12B: Serum antibody titers in rabbits immunized with Formulation II.

### DEFINITIONS

Throughout this invention, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list. (b) all of the items in the list, or (c) any combination of the items in the list. Additionally, the terms “comprising” and the like are used throughout this invention to mean including at least the recited feature(s) such that any greater number of the same feature(s) and/or one or more additional types of features are not precluded. Reference herein to “one embodiment,” “an embodiment,” or similar formulations means that a particular feature of a composition, a composition, a method, or a characteristic described in connection with the embodiment may be included in at least one embodiment of the present technology. Thus, the appearances of such phrases or formulations herein are not necessarily all referring to the same embodiment. Furthermore, various particular features, compositions, methods, or characteristics may be combined in any suitable manner in one or more embodiments.

This invention is not intended to be exhaustive or to limit the present technology to the precise forms disclosed herein, Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain embodiments of the present technology disclosed in the context of particular embodiments may be combined or eliminated in other embodiments. Furthermore, while advantages associated with certain embodiments may have been disclosed in the context of those embodiments, other embodiments may also exhibit such advantages, and not all embodiments need necessarily exhibit such advantages or other advantages disclosed herein to fall within the scope of the present technology. Accordingly, this disclosure and associated technology may encompass other embodiments not expressly shown and/or described herein.

Unless defined otherwise, all technical and scientific terms used herein have the same meaning as commonly understood by one of ordinary skill in the art to which the methods belong. Although any immunogenic compositions, vaccine compositions or methods similar or equivalent to those described herein can also be used in the practice or testing of the embodiments of the present invention, representative illustrative methods and compositions are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods, compositions and combinations.

As used herein, the term “capsular polysaccharide” refers to a layer of polysaccharide external to but contiguous with the cell wall of Streptococcus pneumoniae serotypes 1, 3, 4, 5, 6A, 6B, 7F, 8, 9V, 10A, 11A, 12F. 14, 15A, 18C, 19A, 19F, 22F, 23A, 23B, 23F, 24F, 33F and 35B.

The term “sized” or “sizing” as used herein refers to reducing the size of a native polysaccharide by various methods. The methods may include mechanical methods, such as homogenization. Reducing the size of a native polysaccharide or “sizing” provides various advantages which include: (1) imparting high immunogenicity as compared to the native polysaccharides (2) the ratio of polysaccharide to protein in the conjugate can be altered (3) sized polysaccharides may provide greater stability to the composition.

The term “Molecular weight” or “Molecular size” or “Average Molecular size” or “Average molecular weight” of a polysaccharide as used herein refers to the weight-average molecular weight (Mw) of the polysaccharide measured by MALLS (Multi-Angle Laser Light Scattering).

As used herein, the terms “immunogenic composition” and “vaccine composition” are used interchangeably.

As used herein, the term “carrier protein” refers to any protein or fragment thereof to which the haptens (weak antigens) is coupled or attached or conjugated, typically for the purpose of enhancing or facilitating detection of the antigen by the immune system. Examples of carrier proteins include, but are not limited to CRM₁₉₇, PsaA, Tetanus toxoid and fragments thereof.

The term “conjugate” or “conjugated” as used herein is used to mean that a Streptococcus pneumoniae capsular polysaccharide is covalently bonded to a carrier protein.

As used herein, the term “adjuvant” refers to the non-antigenic component of the vaccine that enhances the immune response of the antigens of the vaccine by facilitating the contact between the antigen and the immune system by influencing the type and the quality of the immune response generated against an antigen. The adjuvant causes prolonged immune responses against the antigens and also may serve to decrease the toxicity of certain antigens or provide solubility to certain antigens.

As used herein, the term “pharmaceutically acceptable carrier(s)” refers to one or more optional components which may be added to the vaccine formulation for administration of the antigens and/or viruses which does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. The term includes one or more excipient, stabilizer, diluents, buffers or surfactants, lyophilization excipient or a combination thereof. By pharmaceutically acceptable or pharmacologically acceptable is meant a material which is not biologically or otherwise undesirable, i.e., the material may be administered to an individual in a formulation or composition without causing any undesirable biological effects or interacting in a deleterious manner with any of the components of the composition in which it is contained.

### DETAILED DESCRIPTION OF THE INVENTION

The present invention provides a 24 valent pneumococcal polysaccharide-protein conjugate vaccine composition comprising polysaccharides from 24 different serotypes of Streptococcus pneumoniae conjugated to one or more carrier proteins.

In an embodiment, the present invention also provides a pneumococcal conjugate vaccine composition comprising capsular polysaccharide from 24 different serotypes of Streptococcus pneumoniae conjugated to a carrier protein, wherein the serotypes comprise 1, 3, 4, 5, 6A, 6B, 7F, 8, 9V, 10A, 11A, 12F, 14, 15A, 18C, 19A, 19F, 22F, 23A, 23B, 23F, 24F, 33F and 35B wherein the carrier protein is selected from CRM₁₉₇ or combination of CRM₁₉₇ and PsaA or combination of CRM₁₉₇ and Tetanus toxoid or combination of PsaA and Tetanus toxoid or combination of CRM₁₉₇, PsaA and Tetanus toxoid.

In an embodiment, the present invention provides a 24 valent pneumococcal polysaccharide-protein conjugate vaccine composition selected from serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B wherein at least thirteen serotypes are conjugated to CRM₁₉₇ and remaining serotypes are conjugated to PsaA.

In a preferred embodiment, the present invention provides a 24 valent pneumococcal polysaccharide-protein conjugate vaccine composition comprising capsular polysaccharide from serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B, wherein capsular polysaccharide from serotypes 1, 4, 5, 6B, 7F, 9V, 14, 18C, 19A, 19F, 22F, 23F and 33F are conjugated to CRM₁₉₇ carrier protein and capsular polysaccharide from serotypes 3, 6A, 8, 10A, 11A, 12F, 15A, 23A, 23B, 24F and 35B are conjugated to PsaA.

In an embodiment, the present invention provides a 24 valent pneumococcal polysaccharide-protein conjugate vaccine composition comprising capsular polysaccharide from different selected from serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B conjugated to CRM₁₉₇ carrier protein.

In an embodiment, the present invention provides a 24 valent pneumococcal polysaccharide-protein conjugate vaccine composition comprising capsular polysaccharide from different selected from serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B conjugated to PsaA carrier protein.

In a preferred embodiment, the present invention provides a 24 valent pneumococcal polysaccharide-protein conjugate vaccine composition comprising capsular polysaccharide from serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B, wherein capsular polysaccharide from serotypes 3, 6A, 8, 10A; 11A, 12F, 15A, 23A, 23B, 24F and 35B are conjugated to PsaA and capsular polysaccharide from serotypes 1, 4, 5, 6B, 7F, 9V, 14, 18C, 19A, 19F, 22F, 23F and 33F are conjugated to combination of CRM₁₉₇ and Tetanus toxoid.

In an embodiment, the present invention provides a pneumococcal vaccine composition comprising pneumococcal polysaccharides wherein one or more of the pneumococcal polysaccharides are native pneumococcal polysaccharides.

In another embodiment, the present invention provides a pneumococcal vaccine composition comprising pneumococcal polysaccharides wherein one or more of the pneumococcal polysaccharides are fragmented, each fragmented pneumococcal polysaccharide having an average molecular weight less than that of a native pneumococcal polysaccharide and may range from 50 to 1000 kDa.

In yet another embodiment, the invention provides an isolated and purified capsular polysaccharides from serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B wherein each polysaccharide having a molecular weight between about 50) and 1000 kDa, preferably, having an average size (Mw) of between 100-1000, 200-800, 250-600, or 300-400, 70-150, or 75-125 kDa.

In yet other embodiments, the present invention provides pneumococcal polysaccharide-protein conjugate vaccine compositions comprising polysaccharides from 24 different serotypes of Streptococcus pneumoniae 1, 3, 4, 5, 6A, 6B, 7F, 8, 9V, 10A, 11A, 12F, 14, 15A, 18C, 19A, 19F, 22F, 23A, 23B, 23F, 24F, 33F and 35B, conjugated to carrier protein selected from CRM₁₉₇ or combination of CRM₁₉₇ and PsaA or combination of CRM₁₉₇ and Tetanus toxoid or combination of PsaA and Tetanus toxoid or combination of CRM₁₉₇, PsaA and Tetanus toxoid, wherein the polysaccharide-protein conjugates having a molecular weight ranging between about 500 kDa to about 5000 kDa; 1,000 kDa to about 10,000 kDa; about 1,500 kDa to about 15,000 kDa; about 2,000 kDa to about 20,000 kDa; about 2,500 kDa to about 25,000 kDa, or about 3,000 kDa to about 30,000 kDa.

In yet another preferred embodiment, the present invention provides a 24 valent pneumococcal polysaccharide-protein conjugate vaccine composition comprising about 2.2 to 2.4 μg of each capsular polysaccharide from serotypes of Streptococcus pneumoniae 1, 3, 4, 5, 6A, 7F, 8, 9V, 10A, 11A, 12F, 14, 15A, 18C, 19A, 19F, 22F, 23A, 23B, 23F, 24F, 33F and 35B and about 4.4 μg 6B, wherein each capsular polysaccharide from serotypes 1, 4, 5, 6B, 7F. 9V, 14, 18C, 19A, 19F, 22F, 23F and 33F are conjugated to about 30 to 35 μg of CRM₁₁₉₇ carrier protein and each capsular polysaccharide from serotypes 3, CA, 8, 10A, 11A, 12F, 15A, 23A, 23B, 24F and 35B are conjugated to about 20 to 30 μg of PsaA.

In yet another preferred embodiment, the present invention provides a 24 valent pneumococcal polysaccharide-protein conjugate vaccine composition comprising about 2.2 to 2.4 μg of each capsular polysaccharide from serotypes of Streptococcus pneumoniae 1, 3, 4, 5, CA, 7F, 8, 9V, 10A, 11A, 12F, 14, 15A, 18C, 19A, 19F, 22F, 23A, 23B, 23F, 24F, 33F and 35B and about 4.4 μg 6B, wherein each capsular polysaccharide is conjugated to about 40 to 80 μg of PsaA.

In yet another preferred embodiment, the present invention provides a 24 valent pneumococcal polysaccharide-protein conjugate vaccine composition comprising about 2.2 to 2.4 μg of each capsular polysaccharide from serotypes of Streptococcus pneumoniae 1, 3, 4, 5, CA, 7F, 8, 9V, 10A, 11A, 12F, 14, 15A, 18C, 19A, 19F, 22F, 23A, 23B, 23F, 24F, 33F and 35B and about 4.4 μg 6B, wherein each capsular polysaccharide is conjugated to about 40 to 80 μg of CRM₁₉₇.

In a further aspect, the present disclosure provides an isolated Streptococcus pneumoniae serotype 15A having an average molecular weight between 50 to 1000 kDa and glycerol content in the range of 5-18%.

The presence of glycerol phosphate side chains can be determined by measurement of glycerol using high performance anion exchange chromatography with pulsed amperometric detection (HPAEC-PAD) after its release by treatment of the polysaccharide with hydrofluoric acid (HF).

In a further aspect, the present disclosure provides an isolated Streptococcus pneumoniae serotype 35B capsular polysaccharide having an average molecular weight between 50 to 1000 kDa and acetate content in the range of 2-10%, preferably 2 to 8%.

In another embodiment, the present invention provides a pneumococcal conjugate vaccine composition comprising pneumococcal polysaccharides where each of the pneumococcal polysaccharides is activated with 1-cyano-4-dimethylamino-pyridinium tetrafluoroborate (CDAP) to form a cyanate ester prior to conjugation to the carrier protein.

In another embodiment, the present invention provides a pneumococcal conjugate vaccine composition comprising pneumococcal polysaccharides where one or more of the pneumococcal polysaccharides are directly coupled to an amino group of the carrier protein or are coupled to the amino group by a spacer.

In another embodiment, the present invention provides a pneumococcal conjugate vaccine composition comprising pneumococcal polysaccharides wherein the spacer is cystamine, cysteamine, hexane diamine, adipic acid dihydrazide (ADH), EDAC or EDC.

PsaA carrier protein according to the present invention is a modified PsaA and does not include wild-type hydrophobic N-terminal leader peptide.

The present invention provides a pneumococcal conjugate vaccine composition comprising pneumococcal polysaccharides of one or more serotypes and a carrier protein wherein the PsaA carrier protein comprise 290 amino acids.

The pneumococcal conjugate vaccine composition comprising capsular pneumococcal polysaccharide serotypes each individually conjugated to a carrier protein, referred to herein as polysaccharide-protein conjugates and/or conjugates. When included in the pneumococcal vaccine composition described herein is a multivalent pneumococcal polysaccharide-protein conjugate vaccine (also referred to herein as multivalent conjugate vaccine, conjugate vaccine, and/or polysaccharide-protein conjugate vaccine). In addition to the multivalent conjugate vaccine, the present invention provides a process for preparing and/or administering the same to a subject in need thereof.

Carrier proteins are non-toxic and non-reactogenic proteins that are obtainable in a sufficient amount and purity. In some embodiments, the present invention provides a pneumococcal conjugate vaccine composition comprising one or more carrier proteins conjugated to one or more Streptococcus pneumoniae polysaccharides (also referred to herein as “pneumococcal polysaccharides”). By conjugating a pneumococcal polysaccharide to a carrier protein, the pneumococcal polysaccharide has increased immunogenicity over the unconjugated pneumococcal polysaccharide.

In some embodiments of the present invention, a combination of the carrier protein used, Which includes two or more carrier proteins, such as PsaA, CRM₁₉₇, Protein D, Diphtheria toxoid and tetanus toxoid (TT).

In another embodiment, the pneumococcal polysaccharide-protein conjugate compositions of the present invention further comprise one or more of the following: pharmaceutically acceptable carrier, a pharmaceutically acceptable diluent, a buffer, a preservative, a stabilizer, an adjuvant, surfactants, solvents, and/or a lyophilization excipient. Suitable buffers include, but not limited to, Tris(trimethamine), phosphate, acetate, borate, citrate, glycine, histidine and succinate and the like. Suitable surfactants include but not limited to Polysorbate-20, Polysorbate-40, Polysorbate-60 (Tween 60), Polysorbate-80 (Tweet) 80), copolymers of ethylene oxide (EO), propylene oxide (PO), and/or butylene oxide (BO), poloxamer 124, poloxamer 188, poloxamer 237, poloxamer 338 and poloxamer 407.octoxynols, sorbitan trioleate (Span 85), and sorbitan monolaurate and the like at a concentration from about 0.001% to about 2%.

The composition of the present invention is formulated in buffered saline solution having a pH in the range from 5.0 to 8.0.

In some embodiments, the pneumococcal polysaccharides may be extracted from one or more microorganisms (e.g. Streptococcus pneumoniae) according to conventional methods. For example, pneumococcal polysaccharides may be prepared according to known procedures. Furthermore, purification of the pneumococcal polysaccharides may be performed according to the procedure described in PCT publication WO 2016/174683 A1.

The extracted pneumococcal polysaccharides may be purified according to conventional methods and may be used in its native form. In other embodiments, the extracted and purified pneumococcal polysaccharides may be fragmented to obtain one or more portions of the pneumococcal polysaccharide, each portion of the pneumococcal polysaccharide having an average molecular weight less than that of the extracted and purified pneumococcal polysaccharides.

In other embodiments, the extracted and purified pneumococcal polysaccharides may be activated prior to conjugation to one or more carrier proteins. For example, the extracted and purified pneumococcal polysaccharides may be activated (e.g., chemically) prior to conjugation to one or more carrier proteins. Each activated pneumococcal polysaccharide may be each individually conjugated to a carrier protein forming a polysaccharide-protein conjugate (e.g., a glycoconjugate). In other embodiments, one or more of the activated pneumococcal polysaccharides may be conjugated to an individual carrier protein. The conjugates may be prepared by known techniques.

In some embodiments, the pneumococcal polysaccharides may be chemically activated and subsequently conjugated to carrier proteins according to known techniques, such as those described in U.S. Pat. Nos. 4,365,170, 4,673,574 and 4,902,506. For example, pneumococcal polysaccharides can be activated by oxidation of a terminal hydroxyl group to an aldehyde with an oxidizing agent, such as periodate (e.g., sodium periodate, potassium periodate, or periodic acid) by random oxidative cleavage of one or more vicinal hydroxyl groups of the carbohydrates and formation of one or more reactive aldehyde groups.

The pneumococcal polysaccharides may also be activated by CDAP (1-cyano-4-di methylamino-pyridinium tetrafluoroborate) and subsequently conjugated to one or more carrier proteins such as PsaA, CRM₁₉₇, PspA, or combination thereof. In other embodiments, pneumococcal polysaccharides activated with CDAP to form a cyanate ester may be directly conjugated to one or more carrier proteins or conjugated using a spacer (e.g., linker). The spacer may couple to an amino group on the carrier protein. In some embodiments, the spacer may be cystamine or cysteamine, which generates a thiolated polysaccharide that may be coupled to the carrier protein through a thioether linkage to a maleimide-activated carrier protein (e.g., using GMBS) or a haloacetylated carrier protein (e.g., using iodoacetimide, ethyl iodoacetimide SLAB, SIA, SBAP, and/or N-succinimidyl bromoacetate. In other embodiments, the cyanate ester is coupled using hexane diamine or adipic acid dihydrazide (ADH) and an amino-derivitized saccharide is conjugated to a carrier protein using carbodiimide (e.g. EDAC or EDC) chemistry via a carboxyl group on the protein carrier, Such conjugates are described in PCT Publication No. WO 93/15760, PCT Publication No. WO 95/08348, PCT Publication No. WO 96/29094, and Chu et al., 1983, Infect. Immunity 40:245-256.

Other suitable activation and/or coupling techniques for use with the polysaccharide-protein conjugates and vaccine compositions of the present invention include the use of carbodiimides, hydrazides, active esters, norborane, p-nitrobenzoic acid, N-hydroxysuccinimide, S NHS, EDC, TSTU, and other methods described in PCT Publication No. WO 98/42721. For example, conjugation may involve a carbonyl linker which may be formed by reaction of a free hydroxyl group of the saccharide with CDI (See Bethell et al., 1979, J. Biol. Chem. 254:2572-4; I-learn et al., 1981, J. Chromatogr. 218:509-18) followed by coupling with a protein to form a carbamate linkage. In some embodiments, the anomeric terminus may be reduced to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

For example, another suitable activation and/or coupling techniques for use with the polysaccharide-protein conjugates and vaccine compositions of the present invention include the following method: sized pneumococcal polysaccharides (e.g., about 6 mL of sized polysaccharide at a concentration of about 10 mg/mL) and CDAP (e.g., about 100 mg/mL in acetonitrile (w/v)) can be mixed in a glass vial in a ratio of about 1 to about 1 (e.g., by stirring for about 1 minute). The pH of the polysaccharide solution may be adjusted as necessary (e.g., to about 9.25 with about 0.2M triethylamine and stirred for 3 min at room temperature). In addition, PsaA (e.g., about 4 mL of a solution having a concentration of about 15 mg/mL) may be added slowly to the activated pneumococcal polysaccharides (e.g., in a ratio of about 1 to about 1 (Ps:Carrier protein)), The pH of the reaction may be adjusted (e.g., to about 9.05 using 0.2M trimethylamine) and the reaction may be continued (e.g., by stirring for 5 hours at room temperature). The reaction mixture may be quenched (e.g., by addition of an excess concentration of glycine).

In some embodiments, the reaction mixture may be diafiltered using a membrane (e.g., a 100 K MWCO membrane) and may be purified by size-exclusion chromatography. The diafiltered and purified fractions may be analyzed using SEC-MALLS, and an anthrone method. The analyzed fractions containing conjugates may be pooled and sterile filtered (e.g., using 0.2 μm filters).

Following conjugation of pneumococcal polysaccharides to one or more carrier proteins, the polysaccharide-protein conjugates may be purified (e.g., enriched with respect to the amount of polysaccharide-protein conjugate) by a variety of techniques. These techniques include, but are not limited to concentration/diafiltration operations, precipitation/elution, column chromatography, and depth filtration. For example, after the conjugates are purified, the conjugates may be compounded to formulate the pneumococcal polysaccharide-protein conjugate compositions of the present invention, which may be used as vaccines.

In some embodiments, the present invention provides a method for preparing a polysaccharide-protein conjugate of the pneumococcal vaccine composition described herein wherein the method further comprises formulating the polysaccharide-protein conjugate into the pneumococcal vaccine composition including an adjuvant, an excipient, and a buffer.

In some embodiments, the present invention provides a method for preparing a polysaccharide-protein conjugate of the pneumococcal vaccine composition described herein wherein the adjuvant is aluminum phosphate.

In some embodiments, the present invention provides a method of preventing or treating a subject in need thereof comprising, administering a pneumococcal vaccine composition described herein to the subject in need thereof.

In some embodiments, the subject has a disease mediated by Streptococcus pneumoniae, such as invasive pneumococcal disease (IPD).

In one embodiment, the subject is a human, such as an infant (less than about 1 year of age), a toddler (about 12 months to about 24 months of age), a young child (about 2 years to about 5 years of age), an older child (about 5 years to about 13 years of age), an adolescent (about 13 years to about 18 years of age), an adult (about 18 years to about 65 years of age), or an elder (more than about 65 years of age).

In some embodiments, the present disclosure provides a method of inducing an immune response comprising administering an immunologically effective amount of the pneumococcal conjugate vaccine composition described herein to a subject.

In one embodiment, method of inducing an immune response comprising administering the pneumococcal conjugate vaccine composition described herein to the subject systemically, subcutaneously, and/or mucosally.

In some embodiments, an amount of each conjugate in a dose of the vaccine compositions of the present invention is in an amount sufficient to induce an immunoprotective response, such as an immunoprotective response without significant, adverse effects. While the amount of each conjugate may vary depending upon the pneumococcal serotype, each dose of the vaccine compositions may comprise about 0.1 μg to about 50 μg of each pneumococcal polysaccharide, about 0.1 μg to about 10 μg, or about 1 μg to about 5 μg of each pneumococcal polysaccharide conjugated to each carrier protein comprising about 1.5 g to about 5 μg of carrier protein.

In another embodiment, the present invention provides a pneumococcal conjugate vaccine composition comprising pneumococcal polysaccharides and carrier proteins, the pneumococcal conjugate vaccine composition having a percent ratio of protein to polysaccharide (protein/PS) of about 0.3 to about 2.0 protein/PS, preferably, 0.5 to 1.5.

In some embodiments, the purified polysaccharides before conjugation have a molecular weight of between 10 kDa and 2,000 kDa. In other such embodiments, the polysaccharide has a molecular weight of between 50 kDa and 2,000 kDa. between 50 kDa and 2,000 kDa; between 50 kDa and 1,750 kDa; between 50 kDa, and 1,500 kDa; between 50 kDa. and 1,250 kDa; between 50 kDa. and 1,000 kDa; between 50 kDa and 750 kDa; between 50 kDa. and 500 kDa; b 100 kDa and 2,000 kDa; between 100 kDa and 2,000 kDa; between 100 kDa and 1,750 kDa; between 100 kDa. and 1,500 kDa; between 100 kDa and 1,250 kDa; between 100 kDa and 1,000 kDa; between 100 kDa and 750 kDa; between 100 kDa. and 500 kDa.

In other embodiments, the present invention provides pneumococcal polysaccharide-protein conjugate vaccine compositions comprising one or more polysaccharide-protein conjugates having a molecular weight ranging between about 1,000 kDa to about 10,000 kDa, about 1,500 kDa to about 15,000 kDa, about 2,000 kDa to about 20,000 kDa, about 2,500 kDa to about 25,000 kDa, or about 3,000 kDa to about 30,000 kDa.

The pneumococcal polysaccharide-protein conjugate vaccine compositions of the present invention may be manufactured using known methods. For example, the pneumococcal polysaccharide-protein conjugate vaccine compositions may be formulated with a pharmaceutically acceptable diluent or vehicle, e.g. water or a saline solution. In addition, the pneumococcal polysaccharide-protein conjugate vaccine compositions may further include one or more of the following: a buffer, a preservative or a stabilizer, polysorbate, an adjuvant such as an aluminum compound, e.g. an aluminium hydroxide, an aluminium phosphate or an aluminium hydroxyphosphate, and/or a lyophilization excipient. Inclusion of any one of the above compounds in the pneumococcal polysaccharide-protein conjugate vaccine compositions of the present invention may be selected as a function of the mode and route of administration to a subject in need thereof and may further be based on standard pharmaceutical practices.

In yet another preferred embodiment, the present invention provides a 24 valent immunogenic composition comprising pneumococcal capsular polysaccharides from serotypes 1, 3, 4, 5, 6A, 6B, 7F, 8, 9V, 10A, 14, 18C, 19A, 19F, 23F, 11A, 12F, 15A, 22F, 23A, 23B, 24F, 33F and 35B each individually conjugated to CRM₁₉₇, wherein the composition has pH from 4 to 7 and comprise: about 4.4 μg/0.5 mL of 6B; about 2.2 to 4 μg/0.5 mL of all other polysaccharides; about 40 to 80 μg/0.5 mL CRM₁₉₇; 0.2 to 2 mg/0.5 mL of aluminum phosphate; about 1 to 10 mlvi succinate buffer; about 0.5 to 25% sodium chloride; 0.002 to 0.2% polysorbate 80; and 4 mg/mL and 10 trig/0.5 mL of 2-phenoxyethanol.

In yet another preferred embodiment, the present invention provides a 24 valent immunogenic composition comprising pneumococcal capsular polysaccharides from serotypes 1, 4, 5, 6B, 7F, 9V, 14, 18C, 19A, 19F, 22F, 23F and 33F conjugated to CRM₁₉₇ carrier protein and capsular polysaccharide from serotypes 3, 6A, 8, 10A, 11A, 12F, 15A, 23A, 23B, 24F and 35B conjugated to PsaA, wherein the composition has pH from 4 to 7 and comprise: about 4.4 vg/0.5 mL of 6B; about 2.2 to 4 μg/0.5 mL of all other polysaccharides; from 20 to 40 μg/0.5 mL CRM₁₉₇; from 20 to 40 μg/0.5 mL PsaA; 0.2 to 2 mg/0.5 ml of aluminum phosphate; about 1 to 10 mM sodium buffer; about 0.5 to 25% sodium chloride; 0.002 to 0.2% polysorbate 80; and 4 mg/mL and 10 mg/0.5 mL of 2-phenoxyethanol.

In yet another preferred embodiment, the present invention provides a 24 valent immunogenic composition comprising pneumococcal capsular polysaccharides from serotypes 1, 3, 4, 5, 6A, 6B, 7F, 8, 9V, 10A, 14, 18C, 19A, 19F, 23F, 11A, 12F, 15A, 22F, 23A, 23B, 24F, 33F and 3513 each individually conjugated to PsaA, wherein the composition has pH from 4 to 7 and comprise: about 4.4 μg/0.5 mL of 6B; about 2.2 to 4 μg/0.5 mL of all other polysaccharides; about 40 to 80 μg/0.5 mL of PsaA; 0.2 to 2 mg/0.5 mL of aluminum phosphate; about 1 to 10 mM succinate buffer; about 0.5 to 25% sodium chloride; 0,002 to 0.2% polysorbate 80; and 4 mg/mL and 10 mg/0.5 mL, of 2-phenoxyethanol.

In some embodiments, the present invention provides a method for preparing a 24 valent pneumococcal polysaccharide-protein conjugate composition comprising pneumococcal polysaccharides selected from serotypes 1, 3, 4, 5, 6A, 6B, 7F, 8, 9V, 10A, 14, 18C, 19A, 19F, 23F, 11A, 12F, 15A, 22F, 23A, 23B, 24F, 33F and 35B wherein the carrier protein is CRM₁₉₇.

The method for preparing the 24 valent pneumococcal polysaccharide-protein conjugate composition comprises the steps of;
(a) individually conjugating each of the twenty-four activated pneumococcal polysaccharides to CRM₁₉₇ carrier protein,
(b) diafiltering and purifying the conjugates using size exclusion chromatography,
(c) analyzing the purified fractions using SEC-MALLS, pooling fractions containing each of the twenty-four conjugates, and filter sterilizing the monovalent conjugate fractions, and
(d) formulating the 24 conjugates obtained in step (c), an adjuvant, one or more excipients, and buffer to prepare the 24 valent pneumococcal polysaccharide-protein conjugate composition.

In some embodiments, the present invention provides a method for preparing a twenty four valent pneumococcal polysaccharide-protein conjugate composition comprising pneumococcal polysaccharides selected from serotypes 1, 3, 4, 5, 6A, 6B, 7F, 8, 9V, 10A, 11A, 12F, 14, 15A, 18C, 19A, 19F, 22F, 23A, 23B, 23F, 24F, 33F and 35B wherein capsular polysaccharide from serotypes 3, 6A, 8, 10A, 11A, 12F, 15A, 23A, 23B, 24F and 35B are conjugated to PsaA and capsular polysaccharide from serotypes 1, 4, 5, 6B, 7F, 9V, 14, 18C, 19A, 19F, 22F, 23F and 33F are conjugated to CRM₁₉₇. The method for preparing the 24 valent pneumococcal polysaccharide-protein conjugate composition comprises the steps of;
(a) individually conjugating polysaccharide from serotypes 3, 6A, 8, 10A, 11A, 12F, 15A, 23A, 23B, 24F and 35B to PsaA and capsular polysaccharide from serotypes 1, 4, 5, 6B, 7F, 9V, 14, 18C, 19A, 19F, 22F, 23F and 33F are to CRM₁₉₇,
(b) diafiltering and purifying the conjugates using size exclusion chromatography,
(c) analyzing the purified fractions using SEC-MALLS, pooling fractions containing each of the twenty-four conjugates, and filter sterilizing the monovalent conjugate fractions, and
(d) formulating the twenty-four conjugates obtained in step (a), an adjuvant, one or more excipients, and buffer into the twenty-four valent pneumococcal polysaccharide-protein conjugate composition.

In some embodiments, the twenty-four-valent pneumococcal polysaccharide-protein conjugate composition may be filtered (e.g., aseptically).

In one embodiment, the pneumococcal polysaccharides are activated utilizing CDAP. In another embodiment, the adjuvant used is aluminum phosphate.

Each conjugate of the 24-valent may be adsorbed separately or together as a mixture onto an aluminium salt such as aluminium hydroxide, aluminium phosphate and the like or mixture of both aluminium hydroxide and aluminium phosphate. The adsorbent may be prepared in situ or may be added during the manufacturing process. The preparation of 24 valent conjugate may be carried out by adding each conjugate to a vessel or container successively or preparing separate solution containing CRM₁₉₇ conjugates (part 1) and PsaA conjugates (part 2) and adding either part 1 to part 2 or vice versa.

The compositions of the present invention may be formulated into a unit dose, for example, a unit dose vial, into a multiple dose, for example, a multiple dose vial, or a pre-filled syringe. The compositions of the present invention may further comprise of one or more preservative(s) selected from thiomersal, 2-phenoxyethanol and the like, in an amount which may range from about 4 mg/mL to about 20 mg/mL.

In some embodiments, the present invention also provides an immunogenic composition (e.g., a vaccine), such as a pneumococcal polysaccharide-protein conjugate composition, administered as a single dose of about 0.5 MI, formulated to contain at least the following: about 2.2 to 4.4 mg of two or more pneumococcal polysaccharide serotypes, about 1 μg to about 10 μg of PsaA per serotype, about 2 μg to about 5 μg of CRM₁₉₇ for each serotype, about 0.2 mg to about 1 mg of an adjuvant (e.g., aluminum phosphate), and one or more excipients (e.g., sodium chloride, and/or a buffer).

Compositions of the present invention may be administered to a subject in need thereof by any number of conventional routes used in the field of vaccines. For example, compositions of the present invention may be administered systemically, such as parenterally (e.g. subcutaneously, intramuscularly, intradermally and/or intravenously) or mucosally (e.g., orally and/or nasally).

In some embodiments, the present invention also provides methods of inducing an immune response in a subject in need thereof to one or more Streptococcus pneumoniae capsular polysaccharides conjugated to one or more carrier proteins. The methods for inducing the immune response comprise administering an immunologically effective amount of the compositions described herein to the subject in need thereof.

According to the methods of the present invention, the subject to whom the compositions described herein is a human, such as an infant (less than about 1 year of age), a toddler (about 12 months to about 24 months of age), a young child (about 2 years to about 5 years of age), an older child (about 5 years to about 13 years of age), an adolescent (about 13 years to about 18 years of age), an adult (about 18 years to about 65 years of age), or an elder (more than about 65 years of age).

As used herein, an “effective amount” of the compositions described in the present disclosure refers to an amount required to elicit an immune response in the subject to which the composition was administered. The immune response is characterized by the presence of one or more Streptococcus pneumoniae antigen-specific antibodies in the host that significantly reduce the likelihood or severity of infection of Streptococcus pneumoniae during a subsequent challenge.

The following examples are provided to illustrate the invention and are merely for illustrative purpose only and should not be construed to limit the scope of the invention.

### Example 1: Preparation of Pneumococcal Capsular Polysaccharide-CRM₁₉₇ Conjugates

Pneumococcal capsular polysaccharide-CRM₁₉₇ conjugates for pneumococcal serotypes 1, 3, 4, 5, 6B, 7F, 9V, 14, 18C, 19A, 19F, 22F, 23F and 33F were prepared as per the procedure described in PCT publication No. WO 2016/207905.

Polysaccharide CRM₁₉₇ conjugates for pneumococcal serotypes 6A, 8, 10A, 11A, 12F, 15A, 23A, 23B, 24F and 35B were prepared as per the procedure mentioned below:

a) Activation and Conjugation of Pneumococcal Polysaccharide Serotype 6A with CRM₁₉₇ Protein Using CDAP Chemistry.

1000 mg (68.5 mL of 14.6 mg/mL concentration) mechanically size reduced polysaccharide serotype 6A and 5.0 mL of CDAP (100 mg/mL in Acetonitrile (w/v)) was mixed in a glass bottle in the ratio of 1.0:0.5 (PS:CDAP) and stirred for 1 minute. The pH of the polysaccharide solution was adjusted to 9.0 with 8.0 mL of 0.2M triethylamine and stirred for 1 minute at room temperature (RT). 1000 mg of CRM₁₉₇ (66.7 mL of 15.0 mg/mL concentration) was added slowly to the activated polysaccharide in a ratio of 1.0:1.0 (PnPs:CRM).

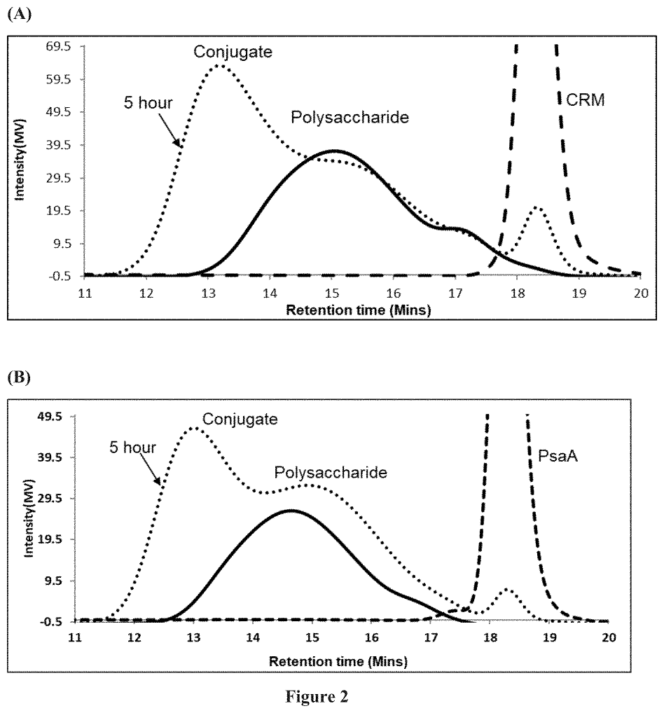

The pH of the reaction was adjusted to 9.0 with 1.0 mL of 0.2M triethylamine and the reaction was continued under stirring for 3-5 hours at room temperature followed by quenching of the reaction by adding excess concentration of glycine (100 mM). The conjugation kinetics (FIG. 2A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, an throne method and fractions containing conjugates were pooled and sterile filtered with 0.2 urn filters.

b) Activation and Conjugation of Pneumococcal Polysaccharide Serotype 8 with CRM₁₉₇ Protein Using CDAP Chemistry.

1000 mg (200.0 mL of 5.0 mg/mL concentration) mechanically size reduced polysaccharide serotype 8 and 4.0 mL of CDAP (100 mg/mL in Acetonitrile (w/v)) was mixed in a glass bottle in the ratio of 1.0:0.4 (PS:CDAP) and stirred for 1 Min. The pH of the polysaccharide solution was adjusted to 9.0 with 7.7 mL of 0.2M triethylamine and stirred for 1 minute at room temperature (RT). 800 mg of CRM₁₉₇ (53.33 mL of 15.0 mg/mL concentration) was added slowly to the activated polysaccharide in a ratio of 1.0:0.8 (PnPs:CRM).

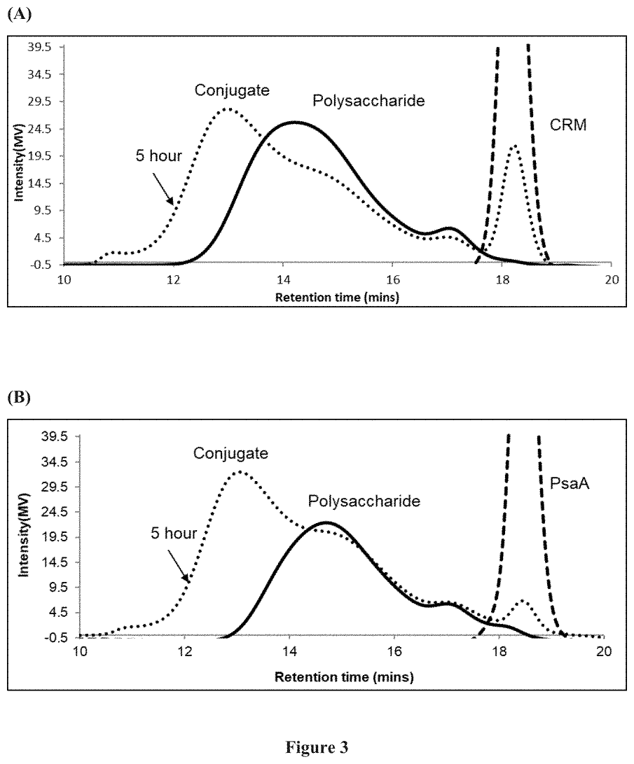

The pH of the reaction was adjusted to 9.0 with 1.5 mL of 0.2M triethylamine and the reaction was continued under stirring for 3-5 hours at room temperature followed by quenching of the reaction by adding an excess concentration of glycine (100 mM). The conjugation kinetics (FIG. 3A) of reactions were monitored using SEC-HPLC at each hour of the reaction.

The reaction mixture was diafiltered and concentrated using 100 kDa, MWCO TFF membrane. The concentrate was purified by size-exclusion chromatography. The fractions were analyzed by SEC-MALLS, anthrone method and fractions containing conjugates were pooled and sterile filtered with 0.2 μm filters.

c) Activation and Conjugation of Pneumococcal Polysaccharide Serotype 10A with CRM₁₉₇ Protein Using CDAP Chemistry.

1000 mg (142.8 mL of 7.0 mg/mL concentration) mechanically size reduced polysaccharide serotype 10A and 8.0 mL of CDAP (100 mg/mL in Acetonitrile (w/v)) was mixed in a glass bottle in the ratio of 1.0:0.8 (PS:CDAP) and stirred for 1 Min. The pH of the polysaccharide solution was adjusted to 9.0 with 13.0 mL of 0.2M triethylamine and stirred for 1 minute at room temperature (RT). 900 mg of CRM₁₉₇ (60.0 mL of 15.0 mg/mL concentration) was added slowly to the activated polysaccharide in a ratio of 1.0:0.9 (PnPs:CRM).

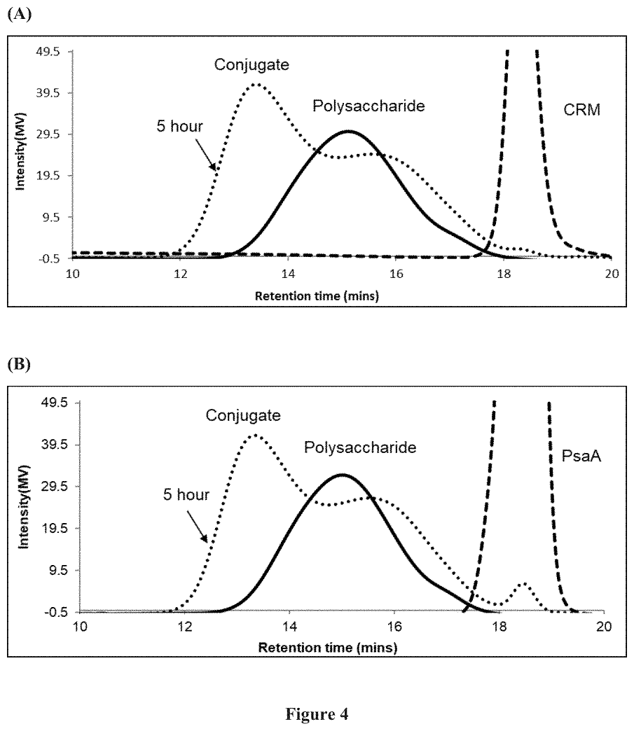

The pH of the reaction was adjusted to 9.0 with 2.5 mL of 0.2M triethylamine and the reaction was continued under stirring for 3-5 hours at room temperature followed by quenching of the reaction by adding an excess concentration of glycine (100 mM). The conjugation kinetics (FIG. 4A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, an throne method and fractions containing conjugates were pooled and sterile filtered with 0.2 μm filters.

d) Activation and Conjugation of Pneumococcal Polysaccharide Serotype 11A with CRM₁₉₇ Protein Using CDAP Chemistry.

1000 mg (125.0 mL of 8.0 mg/mL concentration) mechanically size reduced polysaccharide serotype 11A and 5.0 mL of CDAP (100 mg/mL in Acetonitrile w/v) was mixed in a glass bottle in the ratio of 1.0:0.5 (PS:CDAP) and stirred for 1 minute. The pH of the polysaccharide solution was adjusted to 9.0 with 10.0 mL of 0.2M triethylamine and stirred for 1 minute at room temperature (RT). 1000 mg of CRM₁₉₇ (66.7 mL of 15.0 mg/mL concentration) was added slowly to the activated polysaccharide in a ratio of 1.0:1.0 (PnPs:CRM).

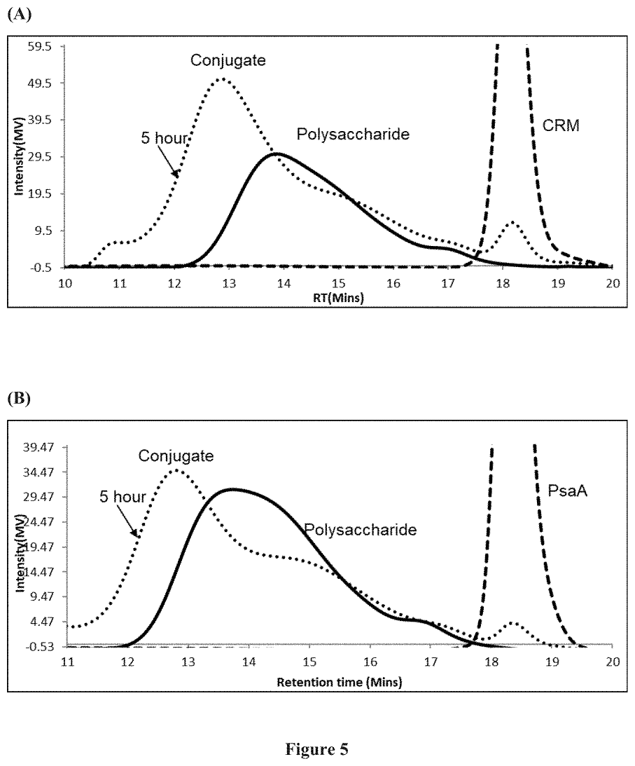

The pH of the reaction was adjusted to 9.0 with 2.5 mL of 0.2M triethylamine and the reaction was continued under stirring for 3-5 hours at room temperature followed by quenching of the reaction by adding an excess concentration of glycine (100 mM). The conjugation kinetics (FIG. 5A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 μm filters.

e) Activation and Conjugation of Pneumococcal Polysaccharide Serotype 12F with CRM₁₉₇ Protein Using CDAP Chemistry.

1000 mg (100.0 mL of 10.0 mg/mL concentration) mechanically size reduced polysaccharide serotype 12F and 5.0 mL of CDAP (100 mg/mL in Acetonitrile (w/v)) was mixed in a glass bottle in the ratio of 1.0:0.5 (PS:CDAP) and stirred for 1 minute. The pH of the polysaccharide solution was adjusted to 9.0 with 10.6 mL of 0.2M triethylamine and stirred for 1 minute at room temperature (RT). 800 mg of CRM₁₉₇ (53.3 mL of 15.0 mg/mL concentration) was added slowly to the activated polysaccharide in a ratio of 1.0:0.8 (PnPs:CRM).

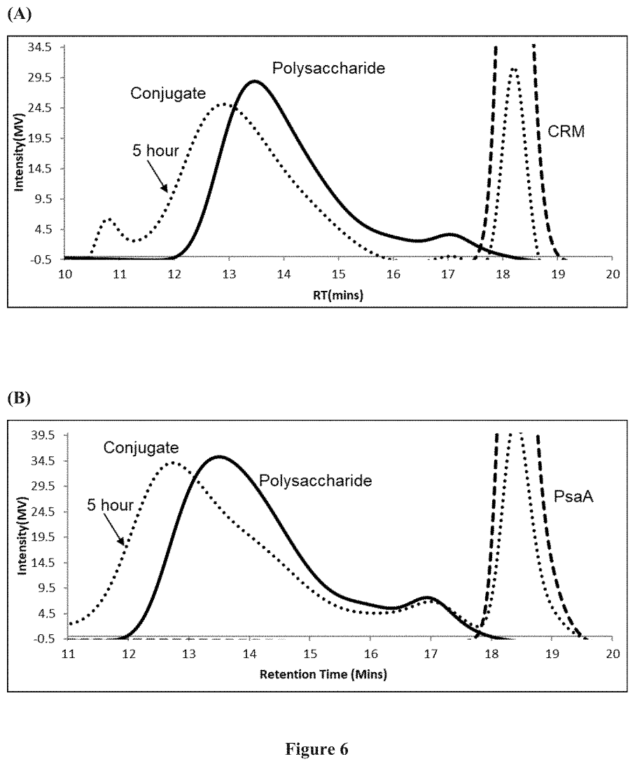

The pH of the reaction was adjusted to 9.0 with 1.0 mL of 0.2M triethylamine and the reaction was continued under stirring for 3-5 hours at room temperature followed by quenching of the reaction by adding an excess concentration of glycine (100 mM). The conjugation kinetics (FIG. 6A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, an throne method and fractions containing conjugates were pooled and sterile filtered with 0.2 urn filters.

f) Activation and Conjugation of Pneumococcal Polysaccharide Serotype 15A with CRM₁₉₇ Protein Using CDAP Chemistry.

1000 mg (66.7 mL of 15.0 mg/mL concentration) mechanically size reduced polysaccharide serotype 15A and 10.0 mL of CDAP (100 mg/mL in Acetonitrile (w/v)) was mixed in a glass bottle in the ratio of 1.0:1.0 (PS:CDAP) and stirred for 1 minute. The pH of the polysaccharide solution was adjusted to 9.0 with 18.0 mL of 0.2M triethylamine and stirred tier 1 minute at room temperature (RT). 1000 mg of CRM₁₉₇ (53.3 mL of 15.0 mg/mL concentration) was added slowly to the activated polysaccharide in a ratio of 1.0:0.8 (PnPs:CRM).

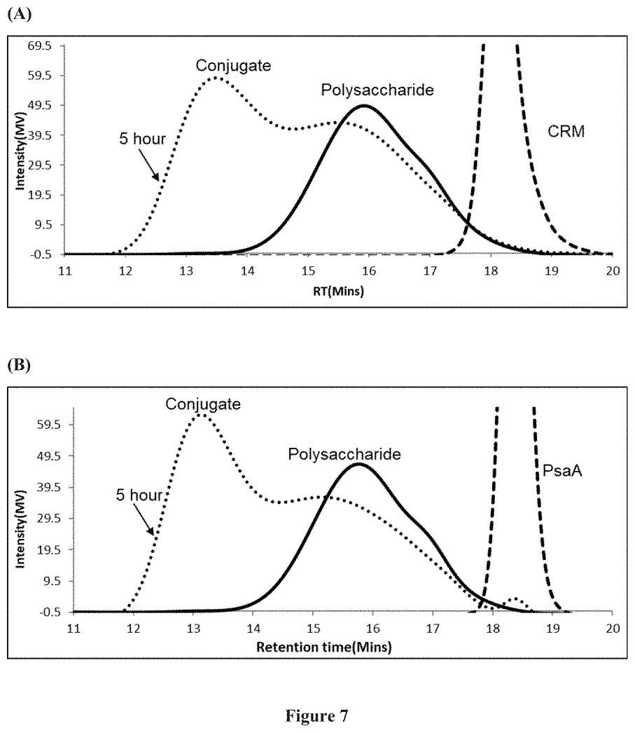

The pH of the reaction was adjusted to 9.0 with 1.0 mL of 0.2M triethylamine and the reaction was continued under stirring for 3-5 hours at room temperature followed by quenching of the reaction by adding an excess concentration of glycine (100 mM). The conjugation kinetics (FIG. 7A) of reactions were monitored using SEC-HPLC at each hour of the reaction.

The reaction mixture was diafiltered and concentrated using 100 kDa, MWCO TFF membrane. The concentrate was purified by size-exclusion chromatography. The fractions were analyzed by SEC-MALLS, anthrone method and fractions containing conjugates were pooled and sterile filtered with 0.2 pm filters.

g) Activation and Conjugation of Pneumococcal Polysaccharide Serotype 23A with CRM₁₉₇ Protein Using CDAP Chemistry.

1000 mg (83.3 mL of 15.0 mg/mL concentration) mechanically size reduced polysaccharide serotype 23A and 10.0 mL of CDAP (100 mg/mL in Acetonitrile (w/v)) was mixed in a glass bottle in the ratio of 1.0:1.0 (PS:CDAP) and stirred for 1 minute. The pH of the polysaccharide solution was adjusted to 9.0 with 14.9 mL of 0.2M triethylamine and stirred for 1 minute at room temperature (RT). 800 mg of CRM₁₉₇ (53.3 mL of 15.0 mg/mL concentration) was added slowly to the activated polysaccharide in a ratio of 1.0:0.8 (PnPs:CRM).

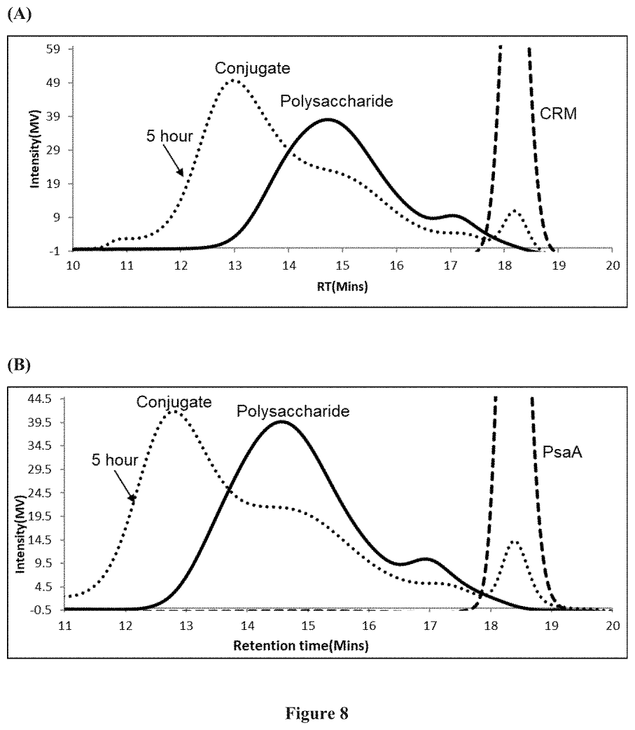

The pH of the reaction was adjusted to 9.0 with 1.7 mL of 0.2M triethylamine and the reaction was continued under stirring for 3-5 hours at room temperature followed by quenching of the reaction by adding an excess concentration of glycine (100 mM). The conjugation kinetics (FIG. 8A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 μm filters.

h) Activation and Conjugation of Pneumococcal Polysaccharide Serotype 23B with CRM₁₉₇ Protein Using CDAP Chemistry.

1000 mg (100.0 mL of 10.0 mg/mL concentration) of mechanically size reduced polysaccharide serotype 23B and 2.0 mL of CDAP (100 mg/mL in Acetonitrile (w/v)) was mixed in a glass bottle in the ratio of 1.0:0.2 (PS:CDAP) and stirred for 1 Min. The pH of the polysaccharide solution was adjusted to 9.0 with 3.5 mL of 0.2M triethylamine and stirred for 1 minute at room temperature (RT). 1000 mg of CRM₁₉₇ (66.7 mL of 15.0 mg/mL concentration) was added slowly to the activated polysaccharide in a ratio of 1.0:1.0 (PnPs:CRM).

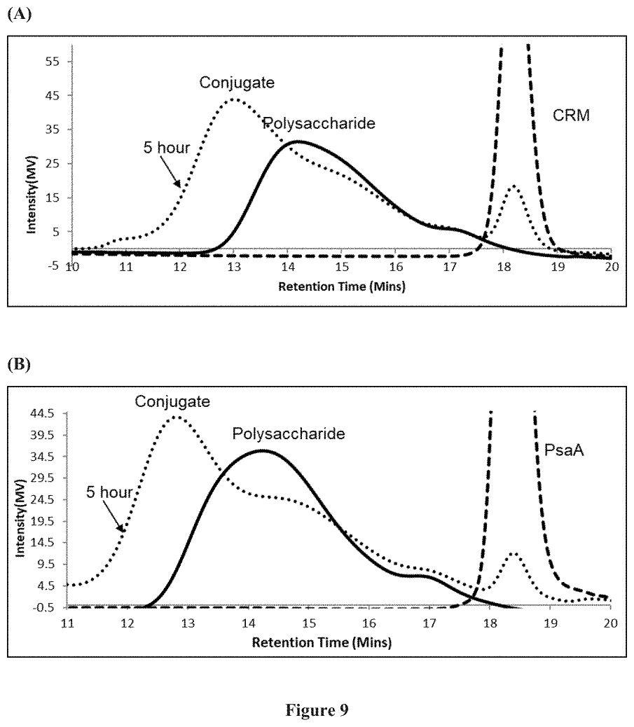

The pH of the reaction was adjusted to 9.0 with 2.2 mL of 0.2M triethylamine and the reaction was continued under stirring for 3-5 hours at room temperature followed by quenching of the reaction by adding an excess concentration of glycine (100 mM). The conjugation kinetics (FIG. 9A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 pin filters.

i) Activation and Conjugation of Pneumococcal Polysaccharide Serotype 24F with CRM₁₉₇ Protein Using CDAP Chemistry.

1000 mg (100.0 mL of 1.0.0 mg/mL, concentration) of mechanically size reduced polysaccharide serotype 24F and 5.0 mL of CDAP (100 mg/mL in Acetonitrile (w/v)) was mixed in a glass bottle in the ratio of 1.0:0.5 (PS:CDAP) and stirred for 1 Min. The pH of the polysaccharide solution was adjusted to 9.0 with 10.6 mL of 0.2M triethylamine and stirred for 1 Min at room temperature (RT). 800 mg of CRM₁₉₇ (53.3 mL of 15.0 mg/mL concentration) was added slowly to the activated polysaccharide in a ratio of 1.0:0.8 (PnPs:CRM).

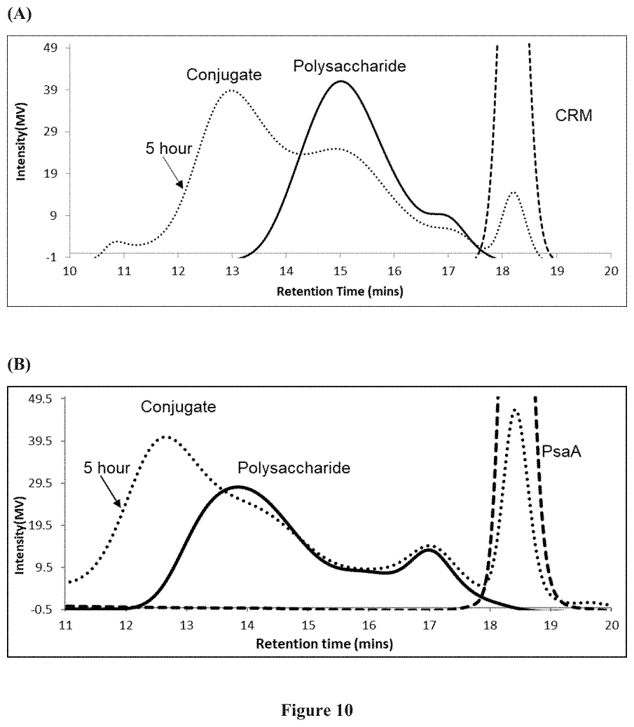

The pH of the reaction was adjusted to 9.0 with 1.0 mL of 0.2M triethylamine and the reaction was continued under stirring for 3-5 hours at room temperature followed by quenching of the reaction by adding an excess concentration of glycine (100 mM). The conjugation kinetics (FIG. 10A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 μm filters.

j) Activation and Conjugation of Pneumococcal Polysaccharide Serotype 35B with CRM₁₉₇ Protein Using CDAP Chemistry.

1000 mg (100.0 mL of 10.0 mg/mL concentration) mechanically size reduced polysaccharide serotype 35B and 5.0 mL of CDAP (100 mg/mL in Acetonitrile (w/v)) was mixed in a glass bottle in the ratio of 1.0:0.5 (PS:CDAP) and stirred for 1 minute. The pH of the polysaccharide solution was adjusted to 9.0 with 5.0 mL of 0.2M triethylamine and stirred for 1 minute at room temperature (RT), 1000 mg of CRM₁ (66.7 mL of 15.0 mg/mL concentration) was added slowly to the activated polysaccharide in a ratio of 1.0:1.0 (PnPs:CRM).

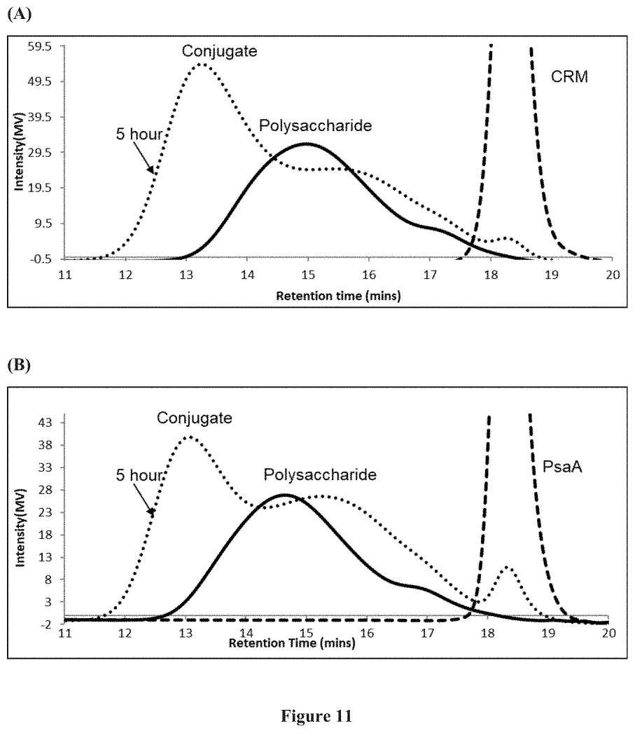

The pH of the reaction was adjusted to 9.0 with 1.6 mL of 0.2M triethylamine and the reaction was continued under stirring for 3-5 hours at room temperature followed by quenching of the reaction by adding an excess concentration of glycine (100 mM). The conjugation kinetics (FIG. 11A) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analysed by SEC-MALLS, anthrone method and fractions containing conjugates were pooled and sterile filtered with 0.2 pm filters.

### Example 2: Preparation of Pneumococcal Capsular Polysaccharide-PsaA Conjugates

A) PsaA Preparation:

The PsaA gene was PCR amplified from Streptococcus pneumoniae Serotype 4, without its hydrophobic leader peptide sequence. The gene sequence was verified and cloned into Escherichia coli using a vector constructed in-house (pBE66) for higher expression.

Glycerol stock culture encoding the PSaA gene was revived on a 20 mL LB Media containing 1 mL of Glycerol Stock in a 150 mL conical flask. The culture was incubated for about 6 hrs at 37° C. under 200 rpm to a final OD 60th of 3.5 OD. The revived culture was transferred to 1 L seed culture in a 5 L conical flask. The culture was grown for about 10 hrs at 37° C. under 200 rpm to a final OD 600 nm of 3. The seed culture was transferred aseptically to a 20 L fermenter containing the following media components. HyPeptone 6 g/L, Yeast extract 12/L, di Potassium Hydrogen ortho phosphate 13.5 gIL, ammonium phosphate di basic 4 g/L Citric acid 1.7 g/L, MgSO₄.7H₂O 1.2 g/L, Glucose 4 g/L, thamine HCL 10 mg/L along with 1 mL/L trace elements (e.g., trace elements for 100 mL composition FeCl₃ 2.7 g, ZnCl₂ 0.2 g, CoCl₂.6H₂O 0.2 g, Na₂MoO₄.2H₂O 0.2 g, CuSO₄ 5H₂O 0.1 g, Boric Acid 0.05 g, CaCl₂) 2H₂O 0.1 g, Conc., HCL 10 mL.) The initial fermentation started with OD₆₀₀ₙₘ 0.2 OD. The pH was maintained at 7±0.2 throughout the fermentation with 20% ortho-phosphoric acid and 12.5% ammonium hydroxide. When the glucose level falls below 0.5 g/L the feed batch was initiated at a steady rate of 3-4 g/L/hr, the DO % was maintained >20% throughout the fermentation with oxygen enrichment. Cells were grown in the fermentor and the cell pellet was harvested by centrifugation. The cells were lysed using cell-disruption device (Panda). The lysate was centrifuged at 10000 g, the clarified supernatant was subject to purification.

PsaA purification was performed similar to the procedure described in Larentis et. al, 2011 (Protein expression and Purification 78 (2011) 38). Purification was further optimized by using mixed mode chromatography (Ceramic Hydroxyapatite Type-II) after DEAE to achieve higher purity of PsaA.

Anion exchange chromatography: 30 mL of DEAE Sepharose (GE) resin was packed in XK16/20 column. The resin was washed with 5 column volumes of sterile distilled water followed by 10 column volumes of 20 mM Tris, 1 mM EDTA, pH 8.0 (Equilibration buffer). 30 mL of supernatant was diluted to 100 mL with equilibration buffer and loaded onto column and flow through was collected. The column was washed with 5 volumes of equilibration buffer. PsaA was eluted with 12 volumes of linear gradient of (0-100% B), (Buffer A containing 20 mM Tris, 1 mM EDTA pH Buffer B-20 mM Tris, 1 mM EDTA, 250 mM NaCl pH 8.0) This was followed by washing the columns with 20 mM Tris, 1 mM EDTA, 1 M NaCl p 8.0.

Mixed mode Chromatography: 25 ml of Ceramic Hydroxyapatite Type II (CHT-II) was packed in column. The resin was washed with volumes of sterile distilled water followed by 10 volumes of 20 mM Tris pH 6.8, Elution fractions from DEAF, resin that showed clear major visible band of approximately 37 KD good concentration of PsaA on SDS PAGE were pooled and loaded onto CHT-II resin. The flow through was collected and the column was washed with 5 column volumes of equilibration buffer. Protein was eluted with 5 column volumes step gradients of (15% B, 20% B, 50% B and 100% B). Buffer A contains 20 mM Tris pH 6.8, while the Buffer B contains 250 mM Phosphate buffer pH 6.8.

All the elution fractions showing a clean band at the expected size of PsaA were pooled, concentrated by 10 kDa. MWCO cassette and diafiltered against 20 mM Phosphate buffer pH 7.5. The purified protein was loaded on SDS-PAGE gel to assess purity.

B) Activation and Conjugation of Pneumococcal Polysaccharide Serotype 3 with PsaA

The size reduced polysaccharide of serotype 3 (concentration of 5 mg/mL) and 1.5 mL of CDAP (100 mg/mL in acetonitrile (w/v)) was mixed in a glass bottle in the ratio of 1:0.5 (PS:CDAP) and stirred for 1 minute. The pH of the polysaccharide solution was adjusted to 9.0 with 3.5 mL of 0.2M triethylamine and stirred for 1 minute at room temperature (RT). 210 mg of PsaA (14.0 mL of 15.0 mg/mL concentration) was added slowly to the activated polysaccharide in a ratio of 1:0.7 (PnPs:PsaA).

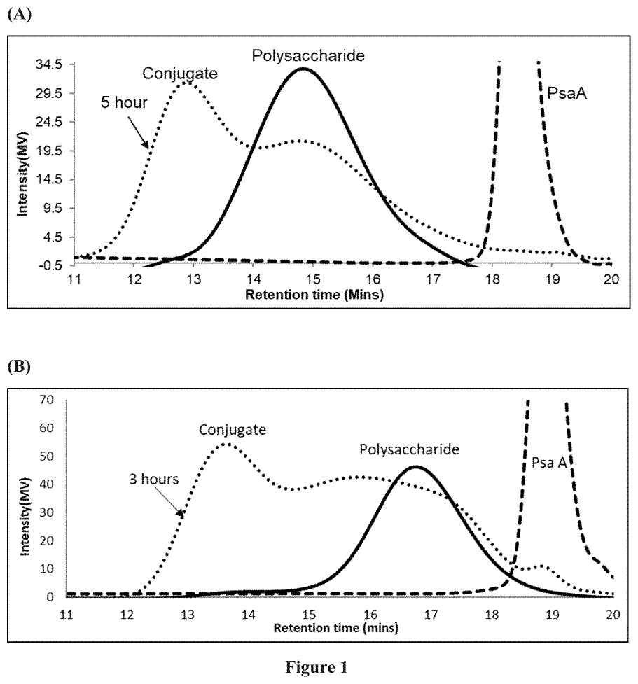

The pH of the reaction was adjusted to about 9.01 with 0.7 mL of 0.2M triethylamine and the reaction was continued under stirring for 5 hours at room temperature followed by quenching of the reaction by adding excess concentration of glycine (100 mM). The conjugation kinetics (FIG. 1A) of reactions were monitored using SEC-HPLC at each hour of the reaction. The reaction mixture was diafiltered and concentrated using 100 kDa, MWCO TFF membrane. The concentrate was purified by size-exclusion chromatography. The fractions were analyzed by SEC-MALLS, anthrone method and fractions containing conjugates were pooled and sterile filtered with 0.2 μm filters.

C) Activation and Conjugation of Pneumococcal Polysaccharide Serotype 6A with PsaA

The size reduced polysaccharide Type 6A (concentration of 14.6 mg/mL) and 400 μL of CDAP (100 mg/mL in Acetonitrile (w/v)) was mixed in a glass bottle in the ratio of 1:1 (PS:CDAP) and stirred for 1 minute. The pH of the polysaccharide solution was adjusted to 9.5 with 800 μL of 0.2M triethylamine and stirred for 1 minute at room temperature (RT), 40 mg of PsaA (3.78 mL of 11.0 mg/mL concentration) was added slowly to the activated polysaccharide in a ratio of 1:1 (PnPs:PsaA).

The pH of the reaction was adjusted to about 9.01 with 0.7 mL of 0.2M triethylamine and the reaction was continued under stirring for 5 hours at room temperature followed by quenching of the reaction by adding excess concentration of glycine (100 mM), The conjugation kinetics (FIG. 2B) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 μm filters.

D) Activation and Conjugation of Pneumococcal Polysaccharide Serotype 6B with PsaA.

The size reduced polysaccharide Type 6B (concentration of 14.97 mg/mL) and 4.0 mL of CDAP (100 mg/mL in acetonitrile (w/v)) was mixed in a glass bottle in the ratio of 1:2 (PS:CDAP) and stirred for 1 minute. The pH of the polysaccharide solution was adjusted to 9.1 with 8.0 mL of 0.2M Triethylamine and stirred for 1 minute at room temperature (RT). 340 mg of PsaA (22.66 mL of 15.0 mg/mL concentration) was added slowly to the activated polysaccharide in a ratio of 1:1.7 (PnPs:PsaA).

The pH of the reaction was adjusted to about 9.01 with 0.7 mL of 0.2M triethylamine and the reaction was continued under stirring for 5 hours at room temperature followed by quenching of the reaction by adding an excess concentration of glycine (100 mM). The conjugation kinetics (FIG. 1B) of reactions were monitored using SECHPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 pm filters.

E) Activation and Conjugation of Pneumococcal Polysaccharide Serotype 8 with PsaA.

1000 mg (200.0 mL of 5.0 mg/mL concentration) of mechanically size reduced polysaccharide serotype 8 and 4.0 mL of CDAP (100 mg/mL in Acetonitrile (w/v)) was mixed in a glass bottle in the ratio of 1.0:0.4 (PS:CDAP) and stirred for 1 minute. The pH of the polysaccharide solution was adjusted to 9.0 with 8.0 nit of 0.2M triethylamine and stirred for 1 minute at room temperature (RT). 800 mg of PsaA (53.33 mL of 15.0 mg/mL concentration) was added slowly to the activated polysaccharide in a ratio of 1.0:0.8 (PnPs:PsaA).

The pH of the reaction was adjusted to 9.0 with 0.1 mL of 0.2M triethylamine and the reaction was continued under stirring for 3-5 hours at room temperature followed by quenching of the reaction by adding an excess concentration of glycine (100 mM). The conjugation kinetics (FIG. 3B) of reactions were monitored using SEC-HPLC at each hour of the reaction.

The reaction mixture was diafiltered and concentrated using 100 kDa. MWCO TFF membrane. The concentrate was purified by size-exclusion chromatography. The fractions were analyzed by SEC-MALLS, anthrone method and fractions containing conjugates were pooled and sterile filtered with 0.2 pm filters.

F) Activation and Conjugation of Pneumococcal Polysaccharide Serotype 10A with PsaA.

1000 mg (142.8 mL of 7.0 mg/mL concentration) of mechanically size reduced polysaccharide serotype 10A and 6.0 al, of CDAP (100 mg/mL in Acetonitrile (w/v)) was mixed in a glass bottle in the ratio of 1.0:0.6 (PS:CDAP) and stirred for 1 minute. The pH of the polysaccharide solution was adjusted to 9.0 with 8.0 mL of 0.2M triethylamine and stirred for 1 minute at room temperature (RT). 800 mg of PsaA (53.33 mL of 15.0 mg/mL concentration) was added slowly to the activated polysaccharide in a ratio of 1.0:0.8 (PnPs:PsaA).

The pH of the reaction was adjusted to 9.0 with 1.3 mL of 0.2M triethylamine and the reaction was continued under stirring for 3-5 hours at room temperature followed by quenching of the reaction by adding an excess concentration of glycine (100 mM). The conjugation kinetics (FIG. 4B) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 pm filters.

G) Activation and Conjugation of Pneumococcal Polysaccharide Serotype 11A with PsaA.

1000 mg (100.0 mL of 10.0 mg/mL concentration) mechanically size reduced polysaccharide serotype 11 A and 8.0 mL of CDAP (100 mg/mL in Acetonitrile (w/v)) was mixed in a glass bottle in the ratio of 1.0:0.8 (PS:CDAP) and stirred for 1 Min. The pH of the polysaccharide solution was adjusted to 9.0 with 14.0 mL of 0.2M triethylamine and stirred for 1 minute at room temperature (RT), 800 mg of PsaA (53.3 mL of 15.0 mg/mL concentration) was added slowly to the activated polysaccharide in a ratio of 1.0:0.8 (PnPs:PsaA).

The pH of the reaction was adjusted to 9.0 with 1.1 mL of 0.2M triethylamine and the reaction was continued under stirring for 3-5 hours at room temperature followed by quenching of the reaction by adding an excess concentration of glycine, (100 mM). The conjugation kinetics (FIG. 5B) of reactions were monitored using SEC-HPLC at each hour of the reaction.

The reaction mixture was diafiltered and concentrated using 100 kDa MWCO TFF membrane. The concentrate was pun fled by size-exclusion chromatography. The fractions were analyzed by SEC-MALLS, anthrone method and fractions containing conjugates were pooled and sterile filtered with 0.2 pm filters.

H) Activation and Conjugation of Pneumococcal Polysaccharide Serotype 12F with PsaA.

1000 mg (142.8 mL of 7.0 mg/al, concentration) mechanically size reduced polysaccharide serotype 12F and 4.0 mL of CDAP (100 mg/mL, in Acetonitrile (w/v)) was mixed in a glass bottle in the ratio of 1.0:0.4 (PS:CDAP) and stirred for 1 Min. The pH of the polysaccharide solution was adjusted to 9.0 with 9.0 mL of 0.2M triethylamine and stirred for 1 minute at room temperature (RT). 700 mg of PsaA (46.6 mL of 15.0 mg/mL concentration) was added slowly to the activated polysaccharide in a ratio of 1.0:0.7 (PnPs:PsaA).

The pH of the reaction was adjusted to 9.0 with 1.7 mL of 0.2M triethylamine and the reaction was continued under stirring for 3-5 hours at room temperature followed by quenching of the reaction by adding an excess concentration of glycine (100 mM). The conjugation kinetics (FIG. 6B) of reactions were monitored using SEC-HPLC at each hour of the reaction.

The reaction mixture was diafiltered and concentrated using 100 kDa. MWCO TFF membrane. The concentrate was purified by size-exclusion chromatography. The fractions were analyzed by SEC-MALLS, anthrone method and fractions containing conjugates were pooled and sterile filtered with 0.2 pm filters.

I) Activation and Conjugation of Pneumococcal Polysaccharide Serotype 15A with PsaA.

1000 mg (71.4 mL of 14.0 mg/mL concentration) mechanically size reduced polysaccharide serotype 15A and 10.0 mL of CDAP (1.00 mg/mL in Acetonitrile (w/v)) was mixed in a glass battle in the ratio of 1.0:1.0 (PS:CDAP) and stirred for 1 Min. The pH of the polysaccharide solution was adjusted to 9.0 with 20.5 mL of 0.2M triethylamine, and stirred for 1 minute at room temperature (RT). 1000 mg of PsaA (66.6 mL of 15.0 mg/mL concentration) was added slowly to the activated polysaccharide in a ratio of 1.0:1.0 (PnPs:PsaA).

The pH of the reaction was adjusted to 9.0 with 0.9 mL of 0.2M triethylamine and the reaction was continued under stirring for 3-5 hours at room temperature followed by quenching of the reaction by adding an excess concentration of glycine (100 mM). The conjugation kinetics (FIG. 7B) of reactions were monitored using SEC-HPLC at each hour of the reaction.

The reaction mixture was diafiltered and concentrated using 100 kDa. MWCO TFF membrane. The concentrate was purified by size-exclusion chromatography. The fractions were analyzed by SEC-MALLS, anthrone method and fractions containing conjugates were pooled and sterile filtered with 0.2 pm filters.

J) Activation and Conjugation of Pneumococcal Polysaccharide Serotype 23A with PsaA.

1000 mg (83.3 mL of 12.0 mg/mL concentration) mechanically size reduced polysaccharide serotype 23A and 10.0 mL of CDAP (100 mg/mL in Acetonitrile (w/v)) was mixed in a glass bottle in the ratio of 1.0:1.0 (PS:CDAP) and stirred for 1 Min. The pH of the polysaccharide solution was adjusted to 9.0 with 20.3 mL of 0.2M triethylamine and stirred for 1 minute at room temperature (RT). 600 mg of PsaA (40.0 mL of 15.0 mg/mL concentration) was added slowly to the activated polysaccharide in a ratio of 1.0:0.6 (PnPs:PsaA).

The pH of the reaction was adjusted to 9.0 with 1.1 mL of 0.2M triethylamine and the reaction was continued under stirring for 3-5 hours at room temperature followed by quenching of the reaction by adding an excess concentration of glycine (100 mM). The conjugation kinetics (FIG. 8B) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, an throne method and fractions containing conjugates were pooled and sterile filtered with 0.2 pm filters.

K) Activation and Conjugation of Pneumococcal Polysaccharide Serotype 23B with PsaA

1.000 mg (100.0 mL of 10.0 mg/mL concentration) mechanically size reduced polysaccharide serotype 23B and 2.0 mL of CDAP (100 mg/mL in Acetonitrile (w/v)) was mixed in a glass bottle in the ratio of 1.0:0.2 (PS:CDAP) and stirred for 1 min. The pH of the polysaccharide solution was adjusted to 9.0 with 3.0 mL of 0.2M triethylamine and stirred for 1 Min at room temperature (RT). 1000 mg of PsaA (66.6 mL of 15.0 mg/mL concentration) was added slowly to the activated polysaccharide in a ratio of 1.0:1.0 (PnPs:PsaA).

The pH of the reaction was adjusted to 9.0 with 2.4 mL of 0.2M triethylamine and the reaction was continued under stirring for 3-5 hours at room temperature followed by quenching of the reaction by adding an excess concentration of glycine (100 mM). The conjugation kinetics (FIG. 9B) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 pm filters.

L) Activation and Conjugation of Pneumococcal Polysaccharide Serotype 24F with PsaA

1000 mg (125.0 mL of 8.0 mg/mL concentration) mechanically size reduced polysaccharide serotype 24F and 3.0 mL of CDAP (100 mg/mL in Acetonitrile (w/v)) was mixed in a glass bottle in the ratio of 1.0:0.3 (PS:CDAP) and stirred for 1 min. The pH of the polysaccharide solution was adjusted to 9.0 with 10.0 mL of 0.2M triethylamine and stirred for 1 Min at room temperature (RT). 600 mg of PsaA (40.0 mL of 15.0 mg/mL concentration) was added slowly to the activated polysaccharide in a ratio of 1.0:0.6 (PnPs:PsaA).

The pH of the reaction was adjusted to 9.0 with 3.5 mL of 0.2M triethylamine and the reaction was continued under stirring for 3-5 hours at room temperature followed by quenching of the reaction by adding an excess concentration of glycine (100 mM). The conjugation kinetics (FIG. 10B) of reactions were monitored using SEC-HPLC at each hour of the reaction.

The reaction mixture was diafiltered and concentrated using 100 kDa MWCO TIFF membrane. The concentrate was purified by size-exclusion chromatography. The fractions were analyzed by SEC-MALLS, anthrone method and fractions containing conjugates were pooled and sterile filtered with 0.2 pm filters.

M) Activation and Conjugation of Pneumococcal Polysaccharide Serotype 35B with PsaA

1000 mg (142.8 mL of 7.0 mg/mL concentration) mechanically size reduced polysaccharide serotype 35B and 6.0 mL of CDAP (100 mg/mL in Acetonitrile (1.1.7/v)) was mixed in a glass bottle in the ratio of 1.0:0.6 (PS:CDAP) and stirred for 1 minute. The pH of the polysaccharide solution was adjusted to 9.0 with 7.0 mL of 0.2M triethylamine and stirred for 1 minute at room temperature (RT). 1000 mg of PsaA (66.6 mL of 15.0 mg/mL concentration) was added slowly to the activated polysaccharide in a ratio of 1.0:1.0 (PnPs:PsaA).

The pH of the reaction was adjusted to 9.0 with 2.2 mL of 0.2M triethylamine and the reaction was continued under stirring for 3-5 hours at room temperature followed by quenching of the reaction by adding an excess concentration of glycine, (100 mM). The conjugation kinetics (FIG. 11B) of reactions were monitored using SEC-HPLC at each hour of the reaction.

The reaction mixture was diafiltered and concentrated using 100 kDa MWCO TFF membrane. The concentrate was purified by size-exclusion chromatography. The fractions were analyzed by SEC-MALLS, anthrone method and fractions containing conjugates were pooled and sterile filtered with 0.2 pm filters.

### Example 3: 24-Valent Pneumococcal Capsular Polysaccharide-Protein Conjugate Vaccine Composition (Formulation I)

A 24 valent conjugated vaccine (0.5 mL) containing 2.2 μg of each pneumococcal polysaccharide from serotypes 1, 4, 5, 7F, 9V, 14, 18C, 19A, 19F, 22F, 23F and 33F and 4.4 μg of serotype 6B, conjugated to about 35 μg CRM₁₉₇; and 2.2 μg of each pneumococcal polysaccharide from serotypes 3, 6A, 8, 10A, 11 A, 12F, 15A, 23A, 23B, 24F and 35B conjugated to about 25 μg of PsaA was prepared in 5 mM succinic acid and about 0.07% w/v polysorbate 20 by adding each conjugate sequentially into blending vessel. To the blended solution, aluminum phosphate gel equivalent to 0.5 mg Al³⁺ per dose of 0.5 mL was added. The pH of the formulation was adjusted to 6.0 using IN hydrochloric acid and under constant stirring. After 2 hours of blending, the formulated blend was aseptically filled at 0.58 mL fill volume per vial into the 3 mL sterile non-siliconized vials, closed with sterile 13 mm rubber stoppers and sealed with 13 mm sterile pink colored flip off aluminum seals, followed by optical inspection and labelling of filled vials. From the lot, some vials were randomly picked and sent for analyzing the appearance, pH, Osmolality, total poly and protein content (μg/SHD), % Adsorption, aluminum content (mg/SHD) (Single Human Dose).

TABLE I
Characterization of Formulation (I)

| | | Total Ps | % | Protein |
|---|---|---|---|---|
| pH | Appearance | (μg/SHD) | Adsorption | (μg/SHD) |
| 6.1 | Whitish Suspension | 52 | 76 | 56 |
| | in which mineral | | | |
| | carrier tends to | | | |
| | settle down slowly | | | |

| Osmolality | Succinic Acid | Polysorbate 20 | Aluminum Content |
|---|---|---|---|
| (mOsmol/kg) | (μg/SHD) | (μg/SHD) | (mg/SHD) |
| 299 | 260 | 300 | 0.52 |

### Example 4: 24-Valent Pneumococcal Capsular Polysaccharide-Protein Conjugate Vaccine Composition (Formulation II)

A 24 valent conjugated vaccine (0.5 mL) containing 2.2 μg of each pneumococcal polysaccharide from serotypes 1.3, 4, 5, 6A, 7F, 8, 9V. 10A, 11A, 12V, 14, 15A, 18C, 19A, 19F, 22F, 23A, 23B, 23F, 24F, 33F and 35B and 4.4 μg of 6B, conjugated to 60 μg CRM₁₉₇ was prepared in 5 mM succinic acid and about 0.07% w/v polysorbate 20 by adding each conjugate sequentially into blending vessel. To the blended solution, aluminum phosphate gel equivalent to 0.5 mg Al³⁺ per dose of 0.5 mL was added. The pH of the formulation was adjusted to 6.0 using IN hydrochloric acid and under constant stirring. After 2 hours of blending, the formulated blend was aseptically filled at 0.58 mL fill volume per vial into the 3 mL sterile non-siliconized vials, closed with sterile 13 mm rubber stoppers and sealed with 13 mm sterile pink colored flip off aluminum seals, followed by optical inspection and labelling of filled vials. From the lot, some vials were randomly picked and sent for analyzing the appearance, pH. Osmolality, total poly and protein content (μg/SHD), % Adsorption, aluminum content (mg/SHD) (Single Human Dose).

TABLE II
Characterization of Formulation (II)

| | | Total Ps | % | Protein |
|---|---|---|---|---|
| pH | Appearance | (μg/SHD) | Adsorption | (μg/SHD) |
| 6.0 | Whitish Suspension | 50 | 78 | 52 |
| | in which mineral | | | |
| | carrier tends to | | | |
| | settle down slowly | | | |

| Osmolality | Succinic Acid | Polysorbate 20 | Aluminum Content |
|---|---|---|---|
| (mOsmol/kg) | (μg/SHD) | (μg/SHD) | (mg/SHD) |
| 293 | 275 | 285 | 0.51 |

### Example 5: Immune Response in Rabbits Immunized with Two Conjugate Vaccines Carrying Different Carrier Proteins

In order to evaluate immunogenicity. Rabbits were immunized with the Formulation I and II. The study design consisted of two groups of 7 rabbits each. Animals were immunized with three doses of each formulation. Bleeding and immunization schedule along with the group details are given in table below:

TABLE III
Assessment of immune response in rabbits to
regular and reduced antigen formulation

Comparators

| | Group | Immunization | |
|---|---|---|---|
| Formulations | size | schedule | Bleeding schedule |
| Formulation I | 7 | 1, 15 and 29 | 0, 14, 28 and 36 |
| Formulation II | 7 | | |

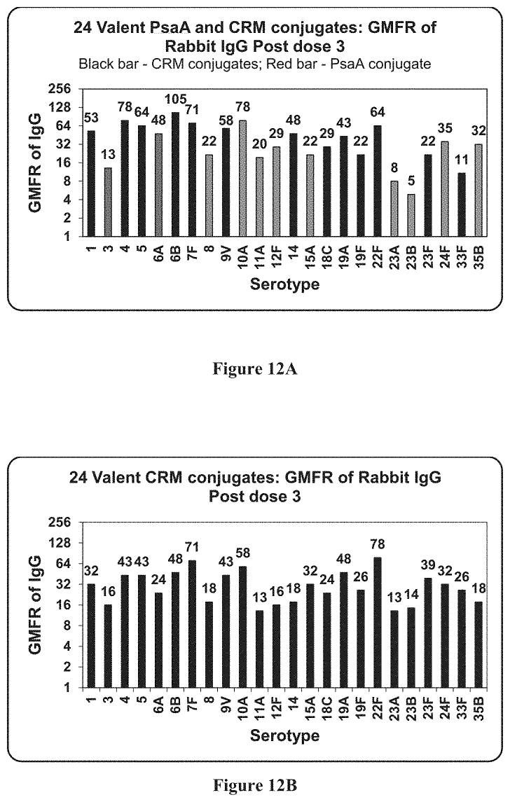

Serum from the immunized rabbits were collected at specified interval. Serotype specific IgG titer levels were estimated in an ELISA, which is adapted from a WHO recommended ELISA to assess serum antibody titers in human serum. Antibody titers were estimated as—maximum dilution of the serum that gave OD₄₅₀ₙₘ value above the cut-off limit. The NG titer value of pre-vaccinated animal was used to calculate Geometric Mean Fold Rise (GMFR) in serum IgG titer. The GMFR titer values were plotted in a graph (FIGS. 12A & 12B).

Titer is estimated as maximum serum dilution that produced ELISA OD₄₅₀ₙₘ (Optical Density at the wavelength of 450 nm) above the cut-off value (2×OD₄₅₀ₙₘ observed in pre-immune sera; OD value of about 0.1. Geometric Mean Fold Rise (GMFR) for each serotype was plotted. The sera obtained after 3 dose of immunization (Post dose 3) was used to assess the immunogenicity. Solid black bars indicate pneumococcal polysaccharides conjugated to CRM₁₉₇, while colored bars indicate pneumococcal polysaccharides conjugated to PsaA.

The serum IgG titers in rabbits vaccinated with either PCV24 (Formulation I) or PCV24 (Formulation II) were found to be very similar. Except Serotypes 23A and 23B in Formulation I immunized group had slightly lower GMFR response when compared to Formulation II. Most importantly all rabbits immunized with either Polysorbate containing one carrier protein (CRM₁₉₇; Formulation II) or two carrier proteins (CRM₁₉₇+PsaA; Formulation I) gave above four-fold rise in GMFR. The four-fold rise in antibody concentration is an acceptance criteria set by WHO for pneumococcal vaccine. Thus, the study shows that there is no negative influence on immune response in presence of another carrier protein on vaccine formulation.

This invention is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain embodiments of the present technology disclosed in the context of particular embodiments may be combined or eliminated in other embodiments. Furthermore, while advantages associated with certain embodiments may have been disclosed in the context of those embodiments, other embodiments may also exhibit such advantages, and not all embodiments need necessarily exhibit such advantages or other advantages disclosed herein to fall within the scope of the present technology. Accordingly, this disclosure and associated technology may encompass other embodiments not expressly shown and/or described herein.

From the foregoing, it will be appreciated that specific embodiments of the invention have been described herein for purposes of illustration, but that various modifications may be made without deviating from the scope of the invention.

### ADVANTAGES OF THE INVENTION

The multivalent pneumococcal conjugate vaccine compositions of the present invention offer an improved immune response over the naive multivalent pneumococcal vaccines and existing pneumococcal conjugate vaccines.

1. A 24 valent pneumococcal conjugate vaccine composition comprising capsular polysaccharide from serotypes of Streptococcus pneumoniae conjugated to a carrier protein, wherein the serotypes comprise 1, 3, 4, 5, 6A, 6B, 7F, 8, 9V, 10A, 11A, 12F, 14, 15A, 18C, 19A, 19F, 22F, 23A, 23B, 23F, 24F, 33F and 35B.

2. The pneumococcal conjugate vaccine composition of claim 1, wherein the carrier protein is selected from CRM₁₉₇ or combination of CRM₁₉₇ and PsaA or combination of CRM₁₉₇ and Tetanus toxoid or combination of PsaA and Tetanus toxoid or combination of CRM₁₉₇, PsaA and Tetanus toxoid.

3. The pneumococcal conjugate vaccine composition of claim 1, wherein the carrier protein is CRM₁₉₇, PsaA or combination thereof.

4. The pneumococcal conjugate vaccine composition of claim 1, wherein capsular polysaccharide from serotypes of Streptococcus pneumonia 1, 3, 4, 5, 6A, 6B, 7F, 8, 9V, 10A, 11 A, 12F, 14, 15A, 18C, 19A, 19F, 22F, 23A, 23B, 23F, 24F, 33F and 35B are conjugated to CRM₁₉₇.

5. The pneumococcal conjugate vaccine composition of claim 1, wherein capsular polysaccharide from serotypes of Streptococcus pneumonia 1, 3, 4, 5, 6A, 6B, 7F, 8, 9V, 10A, 11 A, 12F, 14, 15A, 18C, 19A, 19F, 22F, 23A, 23B, 23F, 24F, 33F and 35B are conjugated to PsaA.

6. The pneumococcal conjugate vaccine composition of claim 1, wherein the vaccine composition is a 24 valent pneumococcal polysaccharide-protein conjugate vaccine composition wherein at least thirteen serotypes are conjugated to CRM₁₉₇ and remaining serotypes are conjugated to PsaA.

7. The pneumococcal conjugate vaccine composition of claim 6, wherein capsular polysaccharide from serotypes 1, 4, 5, 6B, 7F, 9V, 14, 18C, 19A, 19F, 22F, 23F and 33F are conjugated to CRM₁₉₇ and capsular polysaccharide from serotypes 3, 6A, 8, 10A, 11A, 12F, 15A, 23A, 23B, 24F and 35B are conjugated to PsaA.

8. The pneumococcal conjugate vaccine composition of claim 1, wherein capsular polysaccharide from serotypes 3, 6A, 8, 10A, 11A, 12F, 15A, 23A, 23B, 24F and 35B are conjugated to PsaA and capsular polysaccharide from serotypes 1, 4, 5, 6B, 7F, 9V, 14, 18C, 19A, 19F, 22F, 23F and 33F are conjugated to combination of CRM₁₉₇ and Tetanus toxoid or combination of CRM₁₉₇, PsaA and Tetanus toxoid.

9. The pneumococcal vaccine composition claim 1, wherein one or more of the pneumococcal polysaccharides are fragmented, each fragmented pneumococcal polysaccharide has an average molecular weight less than that of a native pneumococcal polysaccharide and range from 50 to 1000 kDa.

10. The pneumococcal vaccine composition of claim 9, wherein the pneumococcal polysaccharides have a molecular weight between about 50 and 1000 kDa, preferably, have an average size (Mw) of between 100-1000, 200-800, 250-600, or 300-400, 70-150, or 75-125 kDa.

11. The pneumococcal vaccine composition claim 1, wherein the polysaccharide-protein conjugates have a molecular weight ranging between 500 kDa to 5000 kDa; 1,000 kDa to 10,000 kDa; 1,500 kDa to 15,000 kDa; 2,000 kDa to 20,000 kDa; 2,500 kDa to 25,000 kDa; or 3,000 kDa to 30,000 kDa.

12. A 24 valent pneumococcal conjugate vaccine composition comprising about 2.2 μg of each capsular polysaccharide from serotypes of Streptococcus pneumoniae 1, 3, 4, 5, 6A, 7F, 8, 9V, 10A, 11A, 12F, 14, 15A, 18C, 19A, 19F, 22F, 23A, 23B, 23F, 24F, 33F and 35B and about 4.4 μg of polysaccharide from serotype 6B, wherein each capsular polysaccharide from serotypes 1, 4, 5, 6B, 7F, 9V, 14, 18C, 19A, 19F, 22F, 23F and 33F are conjugated to about 25 to 40 μg of CRM₁₉₇ carrier protein and each capsular polysaccharide from serotypes 3, 6A, 8, 10A, 11A, 12F, 15A, 23A, 23B, 24F and 35B are conjugated to about 25 to 40 μg of PsaA.

13. A 24 valent pneumococcal conjugate vaccine composition comprising about 2.2 to 2.4 μg of each capsular polysaccharide from serotypes of Streptococcus pneumoniae 1, 3, 4, 5, 6A, 7F, 8, 9V, 10A, 11A, 12F, 14, 15A, 18C, 19A, 19F, 22F, 23A, 23B, 23F, 24F, 33F and 35B and about 4.4 μg 6B, wherein each capsular polysaccharide is conjugated to about 40 to 80 μg of PsaA.

14. A 24 valent pneumococcal conjugate vaccine composition comprising about 2.2 to 2.4 μg of each capsular polysaccharide from serotypes of Streptococcus pneumoniae 1, 3, 4, 5, 6A, 7F, 8, 9V, 10A, 11 A, 12F, 14, 15A, 18C, 19A, 19F, 22F, 23A, 23B, 23F, 24F, 33F and 35B and about 4.4 μg 6B, wherein each capsular polysaccharide is conjugated to about 40 to 80 μg of CRM₁₉₇.

15. The pneumococcal conjugate vaccine composition claim 12, wherein serotype 15A has a glycerol content in the range of 5-18%.

16. The pneumococcal conjugate vaccine composition claim 12, wherein serotype 35B has a glycerol content in the range of 2-10%, preferably 2-8%.

17. The pneumococcal conjugate vaccine composition of claim 1 further comprising one or more of the following: a pharmaceutically acceptable carrier, a pharmaceutically acceptable diluent, a buffer, a preservative, a stabilizer, an adjuvant, and/or a lyophilization excipient.

18. A pneumococcal conjugate vaccine composition of 17, wherein the adjuvant is aluminum phosphate.

19. A method of preventing or treating a subject in need thereof comprising, administering a pneumococcal vaccine composition claim 1 wherein the subject has a disease mediated by Streptococcus pneumoniae, such as invasive pneumococcal disease (IPD).

20. The method of claim 19, wherein the subject is a human, such as an infant (less than about 1 year of age), a toddler (about 12 months to about 24 months of age), a young child (about 2 years to about 5 years of age), an older child (about 5 years to about 13 years of age), an adolescent (about 13 years to about 18 years of age), an adult (about 18 years to about 65 years of age), or an elder (more than about 65 years of age).

21. The method of claim 20, comprising administering the pneumococcal conjugate vaccine composition to the subject as parenterally (e.g. subcutaneously, intramuscularly, intradermally and/or intravenously) or mucosally (e.g., orally and/or nasally).

22. The pneumococcal conjugate vaccine composition claim 1, wherein each dose of the vaccine compositions comprises 0.1 μg to 50 μg of each pneumococcal polysaccharide, 0.1m to 10 or 1 μg to 5 μg of each pneumococcal polysaccharide conjugated to each carrier protein comprising 1.5 μg to about 70 μg of each carrier protein, more preferably comprising 1.5 μg to about 0.5 μg of each carrier protein.

23. A pneumococcal conjugate vaccine composition claim 1, wherein a percent ratio of protein to polysaccharide (protein/PS) is of 0.3 to 2.0 protein/PS, preferably, 0.5 to 1.5.

24. A method for preparing a 24 valent pneumococcal polysaccharide-protein conjugate composition as claimed in claim 4, wherein the method comprises the steps of:
(a) individually conjugating one or more of twenty-four activated pneumococcal polysaccharides to CRM₁₉₇ carrier protein,
(b) diafiltering and purifying the conjugates using size exclusion chromatography,
(c) analyzing the purified fractions using SEC-MALLS, pooling fractions containing each of the twenty-four conjugates, and filter sterilizing the monovalent conjugate fractions, and
(d) formulating the 24 conjugates obtained in step (a), an adjuvant, one or more excipients, and buffer to prepare the 24 valent pneumococcal polysaccharide-protein conjugate composition.

25. The method of claim 5, wherein the method comprises the steps of:
(a) individually conjugating one or more of twenty-four activated pneumococcal polysaccharides to an immunogenic carrier protein selected from a group comprising PsaA and CRM₁₉₇,
(b) diafiltering and purifying the conjugates using size exclusion chromatography,
(c) analyzing the purified fractions using SEC-MALLS, pooling fractions containing each of the twenty-four conjugates, and filter sterilizing the monovalent conjugate fractions, and
(d) formulating the twenty-four conjugates obtained in step (a), an adjuvant, one or more excipients, and buffer to prepare the twenty-four valent pneumococcal polysaccharide-protein conjugate composition.

26. The pneumococcal conjugate vaccine composition of claim 1, wherein the vaccine is formulated into a unit dose vial, a multiple dose vial, or a pre-filled syringe.

27. The pneumococcal conjugate vaccine of claim 26, further comprising one or more preservative(s) selected from thiomersal, 2-phenoxyethanol, in an amount ranges from about 4 mg/mL to about 20 mg/mL.

28. A 24 valent immunogenic composition comprising pneumococcal capsular polysaccharides from serotypes 1, 3, 4, 5, 6A, 6B, 7F, 8, 9V, 10A, 14, 18C, 19A, 19F, 23F, 11 A, 12F, 15A, 22F, 23 A, 23B, 24F, 33F and 35B each individually conjugated to CRM₁₉₇, wherein the composition has pH from 4 to 7 and comprise: about 4.4 μg/0.5 mL of 6B; about 2.2 to 4 μg/0.5 mL of all other serotypes; about 40 to 80 μg/0.5 mL CRM₁₉₇; 0.2 to 2 mg/0.5 mL of aluminum phosphate; about 1 to 10 mM succinate buffer; about 0.5 to 2.5% w/v sodium chloride; 0.002 to 0.2% w/v polysorbate 80; and 4 mg/mL and 10 mg/0.5 mL of 2-phenoxyethanol.

29. A 24 valent immunogenic composition comprising pneumococcal capsular polysaccharides from serotypes 1, 4, 5, 6B, 7F, 9V, 14, 18C, 19A, 19F, 22F, 23F and 33F conjugated to CRM₁₉₇ and capsular polysaccharide from serotypes 3, 6A, 8, 10A, 11 A, 12F, 15A, 23A, 23B, 24F and 35B conjugated to PsaA, wherein the composition has pH from 4 to 7 and comprise: about 4.4 μg/0.5 mL of 6B; about 2.2 to 4 μg/0.5 mL of all other serotypes; from 20 to 40 μg/0.5 mL CRM₁₉₇; from 20 to 40 μg/0.5 mL PsaA; 0.2 to 2 mg/0.5 mL of aluminum phosphate; about 1 to 10 mM succinate buffer; about 0.5 to 2.5% w/v sodium chloride; 0.002 to 0.2% w/v polysorbate 80; and 4 mg/mL and 10 mg/0.5 mL of 2-phenoxyethanol.

30. A 24 valent immunogenic composition comprising pneumococcal capsular polysaccharides from serotypes 1, 3, 4, 5, 6A, 6B, 7F, 8, 9V, 10A, 14, 18C, 19A, 19F, 23F, 11A, 12F, 15A, 22F, 23A, 23B, 24F, 33F and 35B each individually conjugated to PsaA, wherein the composition has pH from 4 to 7 and comprise: about 4.4 μg/0.5 mL of 6B; about 2.2 to 4 μg/0.5 mL of all other serotypes; about 40 to 80 μg/0.5 mL PsaA; 0.2 to 2 mg/0.5 mL of aluminum phosphate; about 1 to 10 mM succinate buffer; about 0.5 to 2.5% w/v sodium chloride; 0.002 to 0.2% w/v polysorbate 80; and 4 mg/mL and 10 mg/0.5 mL of 2-phenoxy ethanol.
